(19) United States
(12) Patent Application Publication
(10) Pub. No.: US 2014/0056850 A1
(43) Pub. Date: Feb. 27, 2014

(54) DICTYOSTELID AMOEBA AND BIOCONTROL USES THEREOF

(71) Applicant: AmebaGone, LLC, Madison, WI (US)

(72) Inventors: Marcin Filutowicz, Madison, WI (US); Katarzyna Dorota Borys, Stoughton, WI (US); Dean Sanders, Madison, WI (US)

(73) Assignee: AmebaGone, LLC, Madison, WI (US)

(21) Appl. No.: 13/972,553

(22) Filed: Aug. 21, 2013

Related U.S. Application Data
(60) Provisional application No. 61/692,101, filed on Aug. 22, 2012.

(51) Int. Cl. A61K 35/68; A01N 63/00

(52) U.S. Cl. CPC A61K 35/68; A01N 63/00
USPC 424/93.1

(57) ABSTRACT

The present invention relates to Dictyostelids myxamoebae of phylum Mycetozoa and uses thereof. In particular, the present invention relates to the use of amoebae, slugs, or their environmentally stable spores to treat microbial infections and other uses.

This application claims priority to U.S. Provisional Application No. 61/692,101, filed Aug. 22, 2012, which is herein incorporated by reference in its entirety.

### FIELD OF THE INVENTION

The present invention relates to Dictyostelid myxamoebae of phylum Mycetozoa and uses thereof. In particular, the present invention relates to the use of amoebae, slugs, or their environmentally stable spores to treat microbial infections and other uses.

### BACKGROUND OF THE INVENTION

Bacterial pathogens are becoming increasingly resistant to multiple antibiotics, rendering what were once considered miracle cures ineffective (Cohen M L. 2000. Nature. 406:762-767). Without these medicines, clinicians must resort to alternative drugs. Often these drugs are less effective than their predecessors, and they have more side effects. Worse, in some instances, alternative drugs are not an option. Pathogens have been isolated that are resistant to all of the Federal Drug Administration's (FDA) approved antibiotics (Mahgoub S, et al. 2002. Infect Control Hosp Epidemiol. 23:477-479). These organisms are intractable pathogens, the harbingers of civilization's return to a pre-antibiotic era and the suffering this era would entail.

Both in its depth and breath, the problem of antibiotic resistance in pathogens is growing. These pathogens quickly arise and spread. To a large degree, this phenomenon is the result of the rapid acquisition and dissemination of genes that confer antibiotic resistance (D'Costa V M, et al. 2006. Science. 311:374-377; Walsh C 2003. Antibiotics: Actions, Origins, Resistance, ASM Press, Washington, D.C.). Bacteria, even of different genera, can share resistance elements through the processes of transformation, transduction, and conjugation (Mazel D & Davies J. 1999. Cell Mol Life Sci. 56:742-754). As a direct consequence of this transfer, antibiotic resistance genes in the environment have become ubiquitous, and pan-antibiotic-resistant pathogens have emerged. Without swift and creative action by the research and development community, infection may once again become the leading cause of suffering and death in the world.

Present day examples of superbugs are MRSA (Methicillin Resistant Staphylococcus Aureus) (McDougal, et al., 2003. J. of Clin. Microb., November, p. 5113-5120 Vol. 41, No. 11) and MDR (multi-drug resistant) Acinetobacter baumannii. Collectively, these organisms are responsible for over forty-percent of all nosocomial infections and over fifty-percent of dermatological infections that require hospitalization (Bassetti M, et al. 2009. Fut. Microbiol. 3:649-660; Frazee B W, et al. 2005. Ann Emerg Med. 45:311-320). All the current oral treatment options for MRSA have drawbacks (Chambers H F & Hegde S S. 2007. Expert Rev Anti Infect Ther. 5:333-335). Linezolid is very expensive, counter indicated for long term therapy, and has notable toxicities including myelotoxicity, lactic acidosis, serotonin syndrome, and peripheral neuropathy (Garazzino S, et al. 2007. Int J Antimicrob Agents. 29:480-483; Garrabou G, et al. 2007. Antimicrob Agents Chemother. 51:962-967; Lawrence K R, et al. 2006. Clin Infect Dis. 42:1578-1583). MRSA are becoming increasingly resistant to tetracyclines, fluoroquinolones, clindamycin, and vancomycin, and these antibiotics are rapidly becoming non-effective treatments (Kaka A S, et al. 2006. J Antimicrob Chemother. 58:680-683). Furthermore, sulfamethoxazole-trimethoprim has recently been shown to have a treatment failure rate of fifty-percent (Proctor R A. 2008. Clin Infect Dis. 46:584-593).

The situation for MDR A. baumannii is also troubling. MDR strains of this organism have been isolated that are resistant to all approved frontline and secondary antibiotics (Maragakis L L & Perl T M. 2008. Clin Infect Dis. 46:1254-1263). Without effective treatments, patients with MRSA or MDR A. baumannii infections have longer periods of hospitalization, increased morbidity, and a greater likelihood of in-hospital death (Bassetti M, et al. 2009. Fut Microbiol. 3:649-660; Frazee B W, et al. 2005. Ann Emerg Med. 45:311-320).

Antibiotic resistance problem is not limited in its scope to medical settings. Antibiotic uses and misuses in veterinary science and in agriculture are a global and rapidly growing issue. For example, “fire blight, caused by Erwinia amylovora, is a major threat to apple and pear production worldwide. Nearly all pear varieties and many of the most profitable apple varieties and horticulturally-desirable rootstocks planted throughout the U.S. are highly susceptible to fire blight. Therefore, most growers apply the antibiotics streptomycin or oxytetracycline one to three times during bloom to prevent growth of E. amylovora. Although streptomycin and oxytetracycline are effective in preventing fire blight on blossoms, their application likely drives antibiotic resistance in the environment and in the food chain. Innovative approaches are desperately needed to reign in fire blight, a disease that has been smoldering in orchards for more than a century and raging out of control over the past decade. An additional societal benefit of non-conventional treatments of fire blight is the elimination of the bulk of antibiotic use in plant agriculture, since greater than 90% of antibiotics applied to plants is for the control of that disease (Johnson, K. B., and Stockwell, V. O. 2000. Biological control of fire blight. Pages 319-337 in: Fire Blight—the Disease and its Causative Agent, Erwinia amylovora, J. L. Vanneste, ed. CAB International, New York).

In the United States, the intracellular gram-positive pathogen Listeria monocytogenes accounts for less than 1% of cases of food-borne illnesses, but around 28% of food-borne deaths (Mead et al., 1999. Emerg. Infect. Dis. 5:607-625). The primary mode of transmission of this pathogen to humans is the consumption of contaminated food (Kathariou, S. 2002. J. Food Prot. 65:1811-1829.; WHO Working Group. 1998. Foodborne listeriosis. Bull. W. H. O 66:421-428.). The organism contaminates food from a variety of environmental sources and food processing facilities. Some strains of L. monocytogenes have been known to persist in the food processing environment for extended periods of time, even more than 10 years (Kathariou, S. 2002. J. Food Prot. 65:1811-1829.; Tompkin, R. B. 2002. J. Food Prot. 65:709-725). In some cases, persistent strains have been responsible for outbreaks of listeriosis. Resistance of Listeria to antimicrobials or sanitizing agents in food processing environments may result from the ability of the cells to form biofilms (Blackman, I. C., and J. F. Frank. 1996. J. Food Prot. 59:827-831; Kumar, C. G., and S. K. Anand. 1998. Int. J. Food Microbiol. 42:9-27.; Wong, A. C. L. 1998. J. Dairy Sci. 81:2765-2770 16, 30). Biofilms of Listeria have been shown to be much more resistant to stress and to sanitizing agents than planktonic cells (Blackman, I. C., and J. F. Frank. 1996. J. Food Prot. 59:827-831; Chavant et al., 2004. FEMS Microbiol. Lett. 236:241-248; Vatanyoopaisarn et al., 2000. Appl. Environ. Microbiol. 66:860-863).

What is needed are new treatments for microbial infections in animals, plants, and contamination of environmental, and industrial settings.

### SUMMARY OF THE INVENTION

The present invention relates to Dictyostelids myxamoebae of phylum Mycetozoa and uses thereof. In particular, the present invention relates to the use of amoebae, slugs, or their environmentally stable spores to treat microbial infections and other uses.

For example, in some embodiments, the present invention provides a method of killing or slowing the rate of growth of a microorganism (e.g., treating a microbial infection), comprising: contacting a microorganism with a composition (e.g., a pharmaceutical composition) comprising one or more species of amoebae, wherein the contacting kills or slows the growth of the microorganism. In some embodiments, the microorganism is a bacteria (e.g., a pathogenic bacteria such as MRSA, multi-drug resistant bacteria or persister cells of a bacteria) or a fungus. In some embodiments, the microorganisms are present in planctonic or biofilm forms. In some embodiments, the microorganism is in or on a subject. For example, in some embodiments, the microorganism is present in a wound, a mucus membrane (e.g., nostril, throat, ocular, rectum, vagina, etc.), a tissue or an organ of the subject. In some embodiments, the wound is at a temperature above the normal body temperature of the subject or is hypoxic. In some embodiments, the microorganism is in or on a plant (e.g., an agricultural or industrial plant). In some embodiments, the composition comprises two or more species of amoebae. The present invention is not limited to a particular strain or species of amoebae. Examples include, but are not limited to, Dictyostelium discoideum (WS-28 and WS-647 and AX3); D. minutum (Purdue 8a); D. mucoroides (Turkey 27, WS-20, WS-142, WS-255); D. mucoroides complex (WS-309); D. purpureum (WS-321.5 and WS-321.7); D. rosarium (TGW-11); D. sphaerocephalum (FR-14); Polysphondylium pallidum (Salvador); P. violaceum (WS-371a) and one unknown isolate (Tu-4-b). In some embodiments, the composition further comprises a non-amoebae anti-microbial agent, along with one or more carriers or other components.

Certain embodiments of the invention provide a method of treating a subject (e.g., a human) infected with a microorganism, comprising: contacting a subject infected with a microorganism with a pharmaceutical composition comprising one or more species of amoebae, wherein the contacting kills the microorganism.

Additional embodiments provide kits, compositions (e.g., pharmaceutical compositions), comprising: one or more species of amoebae; and a carrier (e.g., a pharmaceutically acceptable carrier).

In some embodiments, the present invention provides for the use of a pharmaceutical composition comprising a) one or more species of amoebae; and b) a pharmaceutically acceptable carrier in the treatment of a subject infected with a microorganism.

### DESCRIPTION OF THE FIGURES

FIG. 1 shows a photograph of feeding amoebae.

FIG. 2 shows an electron micrograph showing several stages of amoebic phagocytosis. (Clockwise from the top): Free Klebsiella aerogenes; D. discoideum forms a cup structure and begins to engulf the bacteria. The bacteria, sequestered within a phagosome are digested (image reproduce from Cohen M L. 2000. Nature. 406: 762-767).

FIG. 3 shows a) development stages of soil-borne amoeba and b) lifecycle of D. discoideum (Modified from Science 325:1199).

FIG. 4 shows a photograph of killing of bacteria by amoebae; formation of clearing zones also known as “plaques.”

FIG. 5 shows synergism versus antagonism between various strains of amoebae feeding on K. pneumoniae.

FIG. 6 shows intraspecies variation in amoebic tolerance of hypoxia. Pictured are three tubes containing semi-solid media inoculated with Klebsiella pneumoniae. Tube (A) was an amoebae-free control. Tube (B) was co-inoculated with D. discoideum WI-647. The arrow points to a clear band created as the burrowing amoebae consumed bacteria. Oxygen tension is lower within the medium than at the surface. Tube (C) was co-inoculated with D. discoideum X3. This isolate formed plaques on plates seeded with K. pneumoniae, indicating that the amoebae can feed and are motile, but no band of bacterial clearing was observed in the tube.

FIG. 7 shows growth/sporulation of various amoebae on wild-type (top) and menD mutant (bottom) of S. aureus. Some amoebae not only feed on bacteria on the plate surface but undergo a full development (middle horizontal panel).

FIG. 8 shows feeding of amoebae on Erwinia amylovora grown in SM2 medium “impregnated” with a slice of a pear. As indicated the plate surface was inoculated either with spores or with amoebae. Two amoebae isolates were tested, AX3 and WS 321.7

FIG. 9 shows growth of amoebae at temperatures encountered in skin wounds.

FIG. 10 shows feeding of amoebae on MRSA USA3000 on non-nutrient agar in the presence of serum.

FIG. 11 shows a comparison of the feeding of amoebae on Klebsiella pneumoniae with and without serum.

FIG. 12 shows feeding of amoebae on a menD mutant of S. aureus in presence and absence of serum.

FIG. 13 shows feeding of amoebae on Wide Type Staphylococcus in presence or absence of serum.

FIG. 14 shows feeding of amoebae on natural isolates of virulent strains of Erwinia amylovora (88, 85.1 and A97.1) a causative agent of fire blight in fruit trees and crops.

FIG. 15 shows feeding of amoebae on virulent bacteria of bean disease Pseudomonas syringe 207.2.

FIG. 16 shows zones of feeding of different dictyostelid strains on lawns of MRSA USA300 and Listeria monocytogenes.

FIG. 17 shows that chemical environment of a MRSA USA300 colony-biofilm is conducive to spore germination and destruction by the emerging from spores and multiplying amoebae.

FIG. 18 shows that chemical environment of a MRSA USA300 biofilm established on polycarbonate surface is conducive to the spore germination and destruction by emerging and multiplying amoebae of an axenic strain AX3.

FIG. 19 shows quantification of the speed and efficiency of biofilm destruction by free-living amoebae added (not spores); without and with porcine serum present.

FIG. 20 shows feeding of a temperate climate strain AX3 and a tropical climate strain Salvador on biofilm-encased cells of MRSA USA300 at a human body temperature.

FIG. 21 shows quantitative analysis of a Klebsiella oxytoca's biofilm destruction by 12 different strains of Dictyostelids.

FIG. 22 shows photographs of killing of bacteria by amoebae.

FIG. 23 shows photographs of killing of bacteria by E. amylovora, Salvador, WS 142, and X3 amoebae.

### DEFINITIONS

To facilitate an understanding of the present invention, a number of terms and phrases are defined below.

The term “medical devices” includes any material or device that is used on, in, or through a subject's or patient's body, for example, in the course of medical treatment (e.g., for a disease or injury). Medical devices include, but are not limited to, such items as medical implants, wound care devices, drug delivery devices, birth control and body cavity and personal protection devices. Examples of medical implants include, but are not limited to, urinary catheters, intravascular catheters, dialysis shunts, wound drain tubes, skin sutures, vascular grafts, implantable meshes, intraocular devices, heart valves, and the like. Wound care devices include, but are not limited to, general wound dressings, biologic graft materials, tape closures and dressings, and surgical incision drapes. Drug delivery devices include, but are not limited to, needles, drug delivery skin patches, drug delivery mucosal patches and medical sponges. Body cavity and personal protection devices, include, but are not limited to, tampons, sponges, surgical and examination gloves, toothbrushes, and contact lenses. Birth control devices include, but are not limited to, intrauterine devices (IUDs), diaphragms, and condoms.

The term “therapeutic agent,” as used herein, refers to compositions (e.g., comprising amoebae) that decrease the infectivity, morbidity, or onset of mortality in a subject contacted by a pathogenic microorganism or that prevent infectivity, morbidity, or onset of mortality in a host contacted by a pathogenic microorganism. As used herein, therapeutic agents encompass agents used prophylactically, e.g., in the absence of a pathogen, in view of possible future exposure to a pathogen. Such agents may additionally comprise pharmaceutically acceptable compounds (e.g., adjutants, excipients, stabilizers, diluents, and the like). In some embodiments, the therapeutic agents of the present invention are administered in the form of topical compositions, injectable compositions, ingestible compositions, and the like. When the route is topical, the form may be, for example, a solution, cream, ointment, salve or spray impregnated with spores or amoebae.

As used herein, the term “pathogen” refers to a biological agent that causes a disease state (e.g., infection, cancer, etc.) in a host. “Pathogens” include, but are not limited to, bacteria, fungi, archaea, protozoans, mycoplasma, and other parasitic organisms.

As used herein, the term “microorganism” refers to any species or type of microorganism, including but not limited to, bacteria, archea, fungi, protozoans, mycoplasma, and parasitic organisms. The present invention contemplates that a number of microorganisms encompassed therein will also be pathogenic to a subject.

The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms that are gram negative or gram positive. “Gram negative” and “gram positive” refer to staining patterns with the Gram-staining process that is well known in the art. (See e.g., Finegold and Martin, Diagnostic Microbiology, 6th Ed., CV Mosby St. Louis, pp. 13-15 [1982]). “Gram positive bacteria” are bacteria that retain the primary dye used in the Gram stain, causing the stained cells to appear dark blue to purple under the microscope. “Gram negative bacteria” do not retain the primary dye used in the Gram stain, but are stained by the counterstain. Thus, gram negative bacteria appear red. In some embodiments, the bacteria are those capable of causing disease (pathogens) and those that cause production of a toxic product, tissue degradation or spoilage.

As used herein, the term “fungi” is used in reference to eukaryotic organisms such as the molds and yeasts, including dimorphic fungi.

As used herein, the term “non-human animals” refers to all non-human animals including, but are not limited to, vertebrates such as rodents, non-human primates, ovines, bovines, lagomorphs, porcines, caprines, equines, canines, felines, ayes, etc.

As used herein the term “biofilm” refers to an aggregation of microorganisms (e.g., bacteria) surrounded by an extracellular matrix or slime adherent on a surface in vivo or ex vivo, wherein the microorganisms adopt altered metabolic states. Planktonic cells are innate elements of both the biofilm formation and erosion processes (Costerton J W, Stewart P S, Greenberg E P. Bacterial biofilms: a common cause of persistent infections. Science. 1999; 284(5418):1318-22). As used herein, the term “subject” refers to organisms to be treated by the methods of embodiments of the present invention. Such organisms preferably include, but are not limited to, mammals (e.g., murines, simians, equines, bovines, porcines, canines, felines, and the like), and most preferably includes humans. In the context of the invention, the term “subject” generally refers to an individual who will receive or who has received treatment (e.g., administration of a amoebae of the present invention and optionally one or more other agents) for a condition characterized by infection by a microorganism or risk of infection by a microorganism.

The term “diagnosed,” as used herein, refers to the recognition of a disease by its signs and symptoms (e.g., resistance to conventional therapies), or genetic analysis, pathological analysis, histological analysis, diagnostic assay (e.g., for microorganism infection) and the like.

As used herein the term, “in vitro” refers to an artificial environment and to processes or reactions that occur within an artificial environment. In vitro environments include, but are not limited to, test tubes and cell cultures. The term “in vivo” refers to the natural environment (e.g., an animal or a cell) and to processes or reaction that occur within a natural environment.

As used herein, the term “host cell” refers to any eukaryotic or prokaryotic cell (e.g., mammalian cells, avian cells, amphibian cells, plant cells, fish cells, and insect cells), whether located in vitro or in vivo.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “genome” refers to the genetic material (e.g., chromosomes) of an organism.

As used herein, the term “effective amount” refers to the amount of a therapeutic agent (e.g., an amoeba) sufficient to effect beneficial or desired results. An effective amount can be administered in one or more administrations, applications or dosages and is not intended to be limited to a particular formulation or administration route.

As used herein, the term “co-administration” refers to the administration of at least two agent(s) (e.g., two amoebae) or amoeba and other therapies to a subject. In some embodiments, the co-administration of two or more agents/therapies is concurrent. In some embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “toxic” refers to any detrimental or harmful effects on a cell or tissue as compared to the same cell or tissue prior to the administration of the toxicant.

As used herein, the term “pharmaceutical composition” refers to the combination of an active agent with a carrier, inert or active, making the composition especially suitable for diagnostic or therapeutic use in vivo, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers, and adjuvants (See e.g., Martin, Remington's Pharmaceutical Sciences, 15th Ed., Mack Publ. Co., Easton, Pa. [1975]).

The term “sample” as used herein is used in its broadest sense. A sample may comprise a cell, tissue, or fluids, nucleic acids or polypeptides isolated from a cell (e.g., a microorganism), and the like.

As used herein, the terms “purified” or “to purify” refer, to the removal of undesired components from a sample. As used herein, the term “substantially purified” refers to molecules that are at least 60% free, preferably 75% free, and most preferably 99%, or more, free from other components with which they are usually associated (e.g., bacteria or fungi).

As used herein, the term “modulate” refers to the activity of a compound (e.g., an amoebae) to affect (e.g., to kill or prevent the growth of) a microorganism.

The term “test compound” refers to any chemical entity, pharmaceutical, drug, and the like, that can be used to treat or prevent a disease, illness, sickness, or disorder of bodily function, or otherwise alter the physiological or cellular status of a sample (e.g., infection by a microorganism). Test compounds comprise both known and potential therapeutic compounds. A test compound can be determined to be therapeutic by using the screening methods of the present invention. A “known therapeutic compound” refers to a therapeutic compound that has been shown (e.g., through animal trials or prior experience with administration to humans) to be effective in such treatment or prevention. In some embodiments, “test compounds” are agents that treat or prevent infection by a microorganism.

### DETAILED DESCRIPTION OF THE INVENTION

The present invention relates to Dictyostelid myxamoebae of phylum Mycetozoa and uses thereof. In particular, the present invention relates to the use of amoebae, slugs, or their environmentally stable spores to treat microbial infections and other uses.

The present invention relates to benign myxamoebae (slime molds, cellular slime molds, Dictyostelids) and uses thereof. In particular, the present invention relates to the use of amoebae or their environmentally stable spores (all 150 species of Dictyostelids produce spores Raper K B, Rahn A W. The dictyostelids. Princeton, N.J.: Princeton University Press; 1984; Eichinger L, Rivero-Crespo F. Dictyostelium discoideum Protocols. Totowa, N.J.: Humana Press; 2006.; Bonner J T. Differentiation in social amoebae. Scientific American. 1959; 201:152-62.; Hagiwara H. The taxonomic study of Japanese dictyostelid cellular slime molds. Tokyo: National Science Museum; 1989; Swanson A, Spiegel F, Cavender J. Taxonomy, slime molds, and the questions we ask. Mycologia. 2002; 94:968-9; Swanson A, Vadell E, Cavender J. Global distribution of forest soil dictyostelids J. Biogeo. 2001; 26(1):133-48) to treat microbial infections and other uses.

The social amoebae belonging to the phylum Mycetozoa have been described as primitive eukaryotes that exhibit characteristics found among both protozoans and fungi (Bonner J T. (2009). The social amoebae: the biology of cellular slime molds; Raper et al., (1984) The Dictyostelids). This description can be summarized in an illustration of their asexual life cycle. Each species of dictyostelids has a vegetative phase where, as microscopic unicellular solitary cells feed upon bacteria, grow, and multiply. When the amoebae exhaust their bacterial food source, they enter a social phase in which individual cells stream together to form a multicellular, differentiated, mobile slug (e.g., Dictyostelium discoideum). Since growth occurred at the single-cell stage, its size depends on how many amoebae have entered the aggregate, and slugs will vary in length from about 0.2 to 2 millimeters, a ten fold range, and by the latest estimates the number of amoebae they contain ranges from about 10,000 to 2 million. The slug eventually comes to rest and develops into a macroscopic fruiting body consisting of a stalk with sorocarp. Within the sorocarp are environmentally and temporally stable spores, which are disseminated by the wind, animals, or the forces generated by the sorocarp falling. From each viable spore a single amoeba arises.

There is no definitive evidence that the eumycetozoan dictyostelid myxamoebae cause disease in humans, plants or animals. Pertinent to the practical implications of bacterial predation, dictyostelids are not known to produce toxic debris. In fact, this lack of general toxicity led to the idea of a new anti-cancer strategy in which D. discoideum vesicles are being investigated for the purpose of drug delivery (Tatischeff et al., 2008. J Fluor 18:319-328; Tatischeff I, and A. Alfsen. 2011. J Biomater Nanobiotechnol 2:494-499).

Embodiments of the present invention provide for the use of Dictyostelids myxamoebae in treatment and prevention of microbial infection, in particular, against some of the most tenacious pathogens. For over 0.6 billions of years Dictyostelids have evolved to safely kill a broad range of pathogenic bacteria. They eat pathogens while leaving no toxic debris, they can be applied to wounds, and they do not harm patients. In many ways, myxamoebae are the microscopic equivalents of maggots, which themselves received FDA approval to be marketed for medical use. The benefits of “bio-surgery” are established and include the potential to be used in combination with chemical “small molecule” antibiotics. Combinatorial therapies can reduce the risk of pathogens acquiring and spreading antibiotic resistance. Amoebae offer many of the same advantages as maggots, while their microscopic, spore-forming lifestyle and the parallels to be drawn with phagocytic immune cells make them more appealing, less expensive to make, and more convenient to use. The utility of dictyostelid-based therapy derives from amoebae (e.g., spores or slugs) being an easily transported and applied antibacterial agent, effective against a broad range of pathogens including drug resistant bacteria.

In human medicine, the use of amoebae feeding on bacteria finds use for application at non-sterile sites (e.g., the skin or mucosal surfaces). At these sites, sufficient numbers of amoebae are used to quickly consume pathogenic bacteria. Since amoebae possess the ability to consume wound bacteria, especially including pathogens that are impervious to chemical antibiotics, they further find use as an effective prophylactic, an adjunct to current therapies, or an independent remedy. In some embodiments, amoebae (e.g., slugs or spores) are applied to infected tissue where they quickly reduce the microbial load and, in doing so, promote healing. The patient populations that benefit from this form of therapy are those with, for example, diabetic skin lesions, burns, and surgical or chance wounds. Amoebae further find use in a variety of additional applications. Examples include, but are not limited to, veterinary science, agriculture, food industry and industrial settings (e.g., prevention or remediation of fouling of machine parts, water lines, medical devices, etc.).

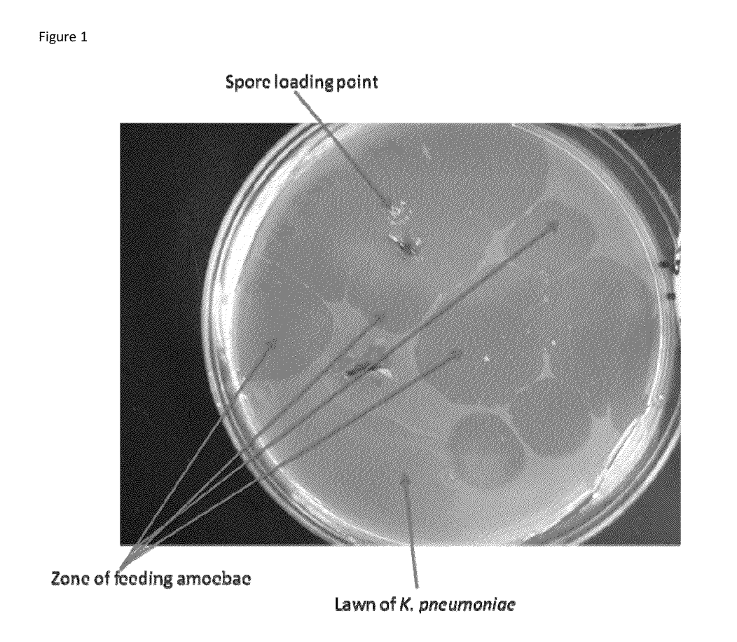

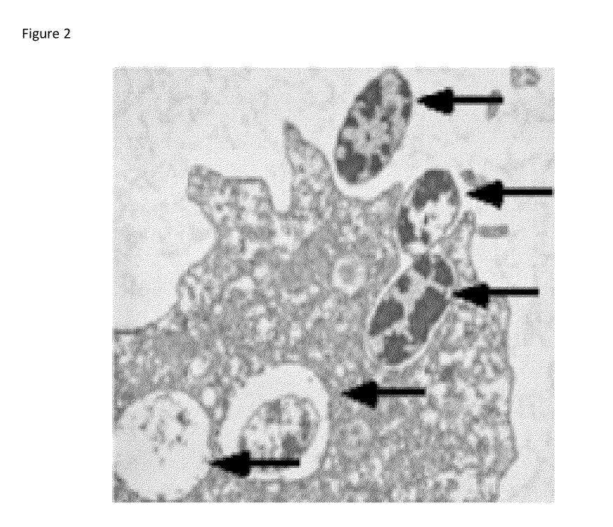

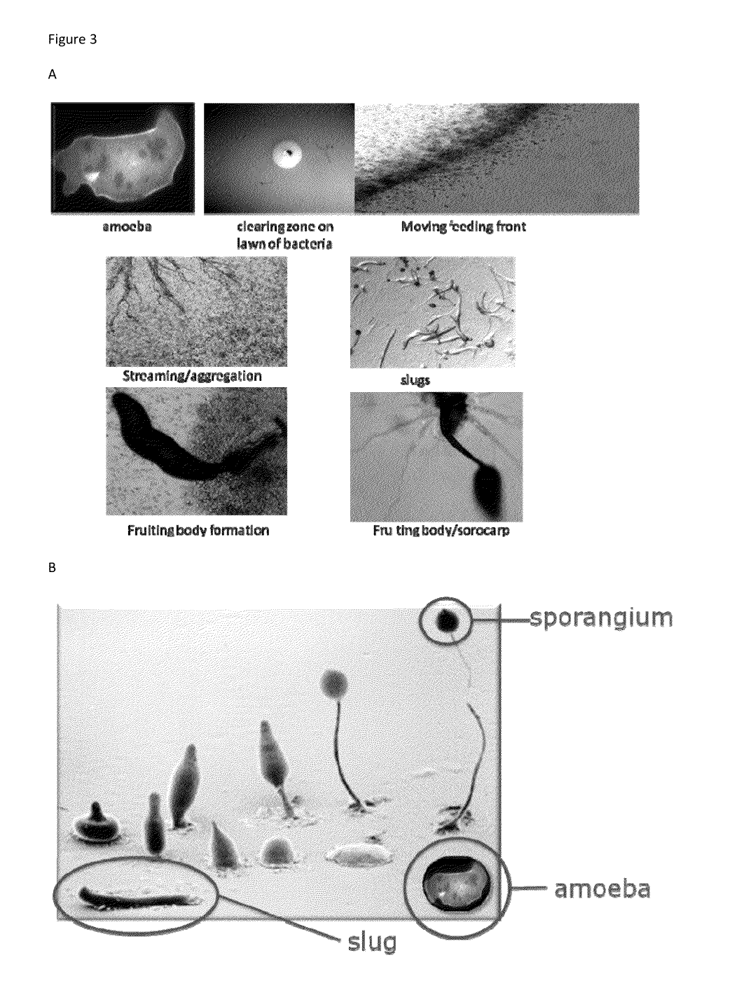

The ability of dictyostelids to feed on bacteria and fungi is described (Raper K B. 1984. The Dictyostelids. Princeton University Press. Princeton N.J.; Old, K. M. et al., 1985 Fine structure of a new mycophagous amoeba and its feeding on Cochliobolus sativus; S. Chakraborty, et al., 1985, Canadian J. of Microb, 31:295-297; Soil Biology and Biochemistry Vol 17, 645-655; A Duczek, L J %A Wildermuth, G B 1991 J Australasian Plant Pathology Vol 20, 81-85). Experiments conducted during the course of developments of embodiments of the present invention demonstrated killing of bacteria spread on a surface of agar plate (FIG. 1). When a few spores are added, in a matter of hours they germinate and from each spore emerges a single amoeba that immediately begins to feed on the surrounding bacteria. As they grow they divide in two (e.g., approximately every three hours) so vast numbers of amoebae are soon present. The free-living amoebae feed first as independent phagocytic cells. Each individual amoeba surrounds a bacterium (or other microorganism) with its pseudopods, encases it in a food vacuole, and extracts the needed nutrients. Thus, amoebae can be viewed as professional phagocytes that are similar to macrophages and neutrophils (Chen G, et al. 2007. Science. 317:678-68). Mechanistically, both amoebae and the immune cells capture bacteria by phagocytosis within cytoplasmic vesicles (FIG. 2). These vesicles fuse with lysosomes as a step in the killing of entrapped bacteria. However, bacterial biofilms are known to be resistant to immune cells (Bjarnsholt et al., Microbiology. 2005;151(Pt 2):373-83; Walker et al., Infection and immunity. 2005; 73(6):3693-701; Mittal et al., Comp Immunol Microbiol Infect Dis. 2006; 29(1):12-26; Jesaitis et al., Journal of immunology. 2003; 171(8):4329-39; Thurlow et al., Journal of immunology. 2011; 186(11):6585-96) but not to majority of Dictyostelids tested in development of embodiments of the present disclosure. Once amoebae clean an area of bacteria, they then come together and aggregate to form a unit similar to a multi-cellular organism. During the social cycle, thousands of non-feeding amoebae aggregate in tune to a camp signal and the community of cells form a slug. Ultimately the slug develops into spore-laden fruiting bodies (FIG. 3). The social amoebae belonging to the phylum Mycetozoa have been described as primitive eukaryotes that exhibit characteristics found among both protozoans and fungi (Bonner J T. (2009); Raper K B, Rahn A W. (1984) The Dictyostelids). This description can be summarized in an illustration of their asexual life cycle (FIG. 3). Each species of amoeba has a vegetative phase where, as microscopic unicellular protists, independent amoeboid cells feed upon bacteria, grow, and multiply. When the amoebae exhaust their bacterial food source, they enter a social phase in which individual cells stream together to form a multicellular, differentiated structures culminating in sporangium (e.g., sorocarp). Within the sporangium are environmentally and temporally stable spores, which are disseminated by the wind, animals, or the forces generated by the sorocarp falling. From each viable spore a single amoeba emerges.

Unlike animals or plants, amoebae eat first; then grow by simply producing an increasing number of separate amoebae, and when food (bacteria/fungi) is gone they stream together to become multi-cellular. Once amoebae form their fruiting bodies they can no longer do anything that requires an intake of energy: they are static. The only part of them that is alive is the dormant spores.

In addition to their feeding behavior, amoebae possess many other virtues that are conducive to an amoebic antimicrobial therapy: Most prominent virtues of this group of organisms have been studied and extensively described for Dictyostelium discoideum. Although the below discussion in exemplified by D. discoideum, the present invention is not limited to a particular strain of amoeba.

D. discoideum amoebae and spores themselves are not known to be pathogenic to animals and plants. D. discoideum consumes and digests a variety of pathogenic and non-pathogenic bacteria, whether live or dead. Moreover, bacteria that are resistant to conventional antibiotics are consumed by D. discoideum (See e.g., Smith M G, et al. 2007. Genes Dev. 21:601-614). D. discoideum not only kills free bacteria, but can consume bacteria living as a colony or biofilm (Raper K B. 1984. The Dictyostelids. Princeton University Press. Princeton N.J.). Thus, dictyostelids further find use in controlling microbial biofilms (e.g., by grazing biofilm-encased cells (e.g., MRSA, USA300, K. pneumoniae, K. oxytoca, E. amylovora). In some embodiments, amoeba are prophylactically administered to patients who are at a high risk of infection (e.g. hospitalized burn patients), that risk unacceptable consequences of infections (e.g. after cosmetic surgery), or who are injured in high risk environments like battlefields. As a eukaryotic organism, D. discoideum amoeba is not susceptible to anti-prokaryotic antibiotics. Therefore, amoebae can be used in conjunction with most of the antibiotics used to treat bacterial infections.

As a phagocytic agent, amoebae internally digest bacteria. Unlike conventional antibiotics, toxic bacterial products are contained and digested within cytoplasmic vesicles. Thus, endotoxic shock reactions seen in patients treated with conventional antibiotics are unlikely following amoebic therapy (Prins J M, et al. 1994. Antimicrob Agent Chemother. 38(6):1211-1218).

In some embodiments, amoebic therapy utilizes overwhelming numbers of amoebae. Locally, these amoebae quickly contain and consume their bacterial prey. The present invention is not limited to a particular mechanism. Indeed, an understanding of the mechanism is not necessary to practice the present invention. Nonetheless, it is contemplated that in the time frame of therapy, resistance to amoebae will be difficult for pathogens to acquire, and spread of resistance will be minimized. Certain bacteria are facultative intracellular pathogens and there are known strain of genetically engineered bacteria, like the benign soil bacterium Bacillus subtilis harboring the gene for lysteriolysin O, can survive within macrophage-like cell line (Bielecki J, et al. 1990. Nature, 345:175-176). However, in combination with more than one amoebae type or in combination with conventional antibiotics, resistance to amoebic therapy can be minimized or eliminated.

### I. Dictystelids

As described above, embodiments of the present invention provide compositions and methods for treating infection by microorganisms with amoebae. Examples of amoebae suitable for use in embodiments of the present invention include, but are not limited to, amoebae of the phylum Mycetozoa, which include but are not limited to:

DICTYOSTELIUM: D. laterosorum, D. tenue, D. potamoides, D. minutum, D. gracile, D. lavandulum, D. vinaceo-fuscum, D. rhizopodium, D. coeruleo-stipes, D. lacteum, D. polycephalum, D. polycarpum, D. polycarpum, D. menorah, D. caveatum, D. gloeosporum, D. oculare, D. antarcticum, D. fasciculatum, D. delicatum, D. fasciculatum, D. aureo-stipes var. helveticum, D. granulophorum, D. medusoides, D. mexicanum, D. bifurcatum, D. stellatum, D. microsporum, D. parvisporum, D. exiguum TNS-C-199, D. mucoroides, D. sphaerocephalum, D. rosarium, D. clavatum, D. longosporum, D. macrocephalum, D. discoideum, D. discoideum AX4, D. intermedium, D. firmibasis, D. brunneum, D. giganteum, D. robustum, D. multi-stipes, Dermamoeba algensis, D. brefeldianum, D. mucoroides, D. capitatum, D. pseudobrefeldianum, D. aureocephalum, D. aureum, D. septentrionalis, D. septentrionalis, D. implicatum, D. medium, D. sphaerocephalum, D. rosarium, D. clavatum, D. longosporum, D. purpureum, D. macrocephalum, D. citrinum, D. dimigraformum, D. firmibasis, D. brunneum, D. giganteum, D. monochasioides, Thecamoeba similis and POLYSPHONDYLIUM: P. violaceum, P. filamentosum, P. luridum, P. pallidum, P. equisetoides, P. nandutensis YA, P. colligatum, P. tikaliensis, P. anisocaule, P. pseudocandidum, P. tenuissimum, P. pallidum, P. asymmetricum, P. filamentosum, P. tenuissimum, P. candidum. ACYTOSTELIUM; A. ellipticum, A. anastomosans, A. longisorophorum, A. leptosomum, A. digitatum, A. serpentarium, A. subglobosum, A. irregularosporum. ACRASIDE; A. granulate, A. rosea; COPROMYXA: C. protea, C. arborescens, C. filamentosa, and C. corralloides; GUTTULINA (Pocheina) G. rosea; GUTTULINOPSIS G. vulgaris, G. clavata, G. stipitata, G. nivea (See e.g., Schaap, et al. 2006 Molecular Phylogeny and Evolution of Morphology in the Social Amoebas, Science 27 Oct. 2006: 661-663; Raper K B. 1984. The Dictyostelids. Princeton University Press. Princeton N. J.; each of which is herein incorporated by reference in its entirety).

Experiments conducted during the course of developments of embodiments of the present invention identified strains of soil-borne amoeba that reduce the bacterial loads Dictyostelium discoideum (WS-28 and WS-647 and X3); D. minutum (Purdue 8a); D. mucoroides (Turkey 27, WS-20, WS-142, WS-255); D. mucoroides complex (WS-309); D. purpureum (WS-321.5 and WS-321.7); D. rosarium (TGW-11); D. sphaerocephalum (FR-14); Polysphondylium pallidum (Salvador); P. violaceum (WS-371a) and one unknown isolate (Tu-4-b) (all names, except D. discoideum X3, given by K. Raper in his collection of slime molds maintained by and available at the Department of Bacteriology at University of Wisconsin-Madison, USA). In this collection a dichotomous key based on cellular morphology and behavior plus the shape and color of spores, sori, or sorocarp has been used to determine the genus and species of the Mycetozoa (Raper K B, Rahn A W. (1984) The Dictyostelids; Swanson A, Spiegel F, Cavender J. (2002) Mycologia 94: 968-9). Despite origins dating back to the early 1900s, this key holds up remarkably well when amoebae are examined using modern molecular techniques. For example, a multiple loci DNA sequence comparison revealed extensive genetic variation among Dictyostelid species (Schaap et al., (2008) Molecular phylogeny and evolution of morphology in the social amoebas. Science 314(5799): 661-3). In addition to confirming the ontological method of classifying the social amoeba, these differences indicate that different species can have unique genetic traits.

The amoebae described herein have evolved to consume a myriad of species of bacteria that live in soil communities. Like macrophages and neutrophils, single celled amoebae chase, engulf and digest their microbial prey (Chen G, Zhuchenko O, Kuspa A. (2007) Science 317(5838): 678-81). Amoebae readily consume planktonic bacteria. In addition, they have acquired the ability to eat bacteria within biofilms within which many soil-dictyostelid amoebae can thrive. Three-dimensional quantification of soil biofilms using image analysis has been performed and it revealed that these biofilms form biologically complex and environmentally harsh soil bio-webs (Rodriguez S, Bishop P. Three-dimensional quantification of soil biofilms using image analysis. Environ Eng Sci. 2007; 24(2):96-103).

The existence of soil amoebae has been known for almost one hundred and fifty years (Brefeld O. (1869) Abh. Seckenberg Naturforsch. Ges. 7: 85-107). But it was not until 1965, when Cavender and Raper (Cavender J C, Raper K B. (1965) Am J Bot 52: 294-6) developed a quantitative method for their enumeration, that extensive ecological studies of these organisms were undertaken. For the best-characterized genus, Dictyostelium, nine species were found to be common inhabitants of the upper soil and leaf litter layers in the forests of North America (Cavender J C, Raper K B. (1965) The Acrasieae in nature. I. Isolation. Am J Bot 52: 294-6). Since the publication of these early studies, it has been shown that the Dictyostelids occur worldwide in a variety of soil environments (Swanson A, Vadell E, Cavender J. (2001) Global distribution of forest soil dictyostelids J Biogeo 26(1): 133-48). Collectively, the ecological studies indicate that amoebae are truly cosmopolitan both with regard to their geographic distribution and ecological niches.

In some embodiments, D. discoideum isolates are utilized. Strains of amoebae have been isolated that grow on bacteria and on synthetic media (Sussman M, 1966. Biochemical and genetic methods in the study of cellular slime mold development. pp. 397-410. In: Methods in Cell Physiology, Vol. 2, Edited by D Prescott. Academic. Press, New York). High numbers of organisms are easily obtained; Chemical and transposon mutagenesis is routinely used with amoebae to isolate growth and functional mutants (Liwerant I J & Pereira da Silva L H. 1975. Mutat Res. 33(2-3):135-46); Barclay S L & Meller E 1983. Mol Cell Biol. 3:2117-2130). D. discoideum is a haploid easing the genetic characterization of mutant organisms; the genome sequence of D. discoideum has been determined and published (Eichinger L, et al. 2005. Nature. 435:43-57). Also, that genome was recently compared to the genome of the genomes sequence of D. purpureum (R. Sucgang et al., 2011, Genome Biology 2011, 12).

In some embodiments, amoeba therapy utilizes D. discoideum isolate AX3, but is not limited to this axenic strain. AX3 isolate has the novel, and useful, property of axenic growth; that is, growth on media without a bacterial food supply. Historically, AX3 is pre-dated by other axenic mutants. Repeated sub-culturing of wild type D. discoideum in a liquid medium containing salts, liver extract, and fetal calf serum was used to obtain archetype axenic strains. Using this technique, Sussman and Sussman isolated AX-1, the first reported axenic mutant (Sussman R & Sussman M. 1967. Biochem. Biophys. Res. Commun. 29:53-55). Based the previous studies, Loomis isolated a N-methyl-N′-nitro-N-nitrosoguanidine (MNNG) mutant that is capable of axenic growth in chemically defined media (Loomis, W F Jr. 1971. EXDI Cell Res. 64, 484-486). This strain, named AX3, has at three genetically-defined mutations that confer the growth phenotype (Williams K L et al. 1974. Nature, London 247, 142-143; North M J & Williams K L. 1978. J. Gen. Microbiol. 107:223-230). As a basis for amoebic therapy, propagation of D. discoideum in bacteria-free cultures is a strong advantage. Axenic cultures can be used to manufacture the large numbers of pathogen-free amoebae or spores that are needed for therapy.

Advantageous phenotypes can be linked to multiple genetic mutations, and these mutations can be serially selected using multiple rounds of MNNG mutagenesis. Most questions concerning amoebic therapy can be addressed by manipulating of the amoeba's genome. Amoeba can be genetically altered by chemical mutagenesis or with molecular techniques. For example, D. discoideum is a haploid organism. Its genome sequence is published and mutants are easily generated by chemical mutagenesis, gene replacement technologies, and by RNA interference (Barclay S L & Meller E. 1983. Mol Cell Biol. 3:2117-2130; Eichinger L, et al. 2005. Nature. 435:43-57).

In some embodiments, amoebae are stored and/or transported in the spore stage of the life cycle. D. discoideum forms easily germinated temperature-, environment-, and temporally-stable spores. In the absence of a bacterial food supply, essential amino acids become limiting, and D. discoideum sporulates. Spores have been shown to remain viable, without refrigeration, for over 50 years when lyophilized. When nutrients are available, spores germinate in 3-10 hours to produce amoebae. Spores can be exploited as a means of transport and storage of medicinal amoebae. For convenience, spores can be administered embedded in bandages or dressings, gels, etc.

In some embodiments, amoebae are stored/transported in the slug stage of the life cycle. Slugs are able to exploit new territories for food as they move through the medium (Kuzdzal-Fick et al., (2007) Behav Ecol 18(2): 433-7). D. discoideum slugs have been observed to be continuously shedding amoebae (Smith E, Williams K L. (1979) FEMS Microbiol Lett. 6: 119-22; Morrissey J H. (1982) Cell proportioning and pattern formation. The Development of Dictyostelium discoideum.: 411-43; Sternfeld J. (1992) Roux's Arch Dev Biol. 201: 354-63; Wilkins M R, Williams K L. (1995) Experientia 51(12): 1189-96; Alexander R D. (1974) Annu Rev Ecol Syst. 5: 325-83; Raper K B. (1956) Mycologia 48(2)160-205; Chen et al., Science. 2007; 317(5838):678-81) Each slug acts as a mobile distributor of cells to local areas. Another species of slime mold, D. polycephalum, also loses amoebae from migrating slugs (Bonner J T. Migration in Dictyostelium polycephalum. Mycologia. 2006; 98(2):260-4). The mentioned properties of the slugs serve as a method of local dispersal of amoebae to food patches. Alternatively, mechanically disaggregated slug cells (most of which are non feeding) are deployed as they are able to dedifferentiate from aggregates to become solitary feeding amoebae (Katoh et al., (2004) Proc Natl Acad Sci USA 101(18): 7005-10). Thus, the occurrence of dedifferentiation means that slugs are able to breakup on contact with a new food source. Solitary amoebae move more slowly and travel much shorter distances than slugs. For example, aggregating cells generally travel 1 cm at most, in contrast, slugs traveling on agar (and through soil) can cover distances of 10-20 cm in a matter of days (Kessin R H. Dictyostelium: evolution, cell biology, and the development of multicellularity. Cambridge, UK; New York: Cambridge University Press; 2001; Bonner J T. Mycologia. 2006; 98(2):260-4). Thus, in some embodiments, the slug's migratory properties are used to deposit amoebae at sites that solitary amoebae may have difficulty reaching.

In some embodiments, the present invention provides kits and/or compositions comprising amoebae. In some embodiments, amoebae are in a form (e.g., spores, aspidocytes (Serafimidis et al., Microbiology. 2007; 153(Pt 2):619-30) or slugs) that is stable for long term storage. In other embodiments, amoebae are stored and transported in different stages. In some embodiments, compositions comprise additional components (e.g., storage reagents, buffers, preservatives, stabilizers, etc.). In some embodiments, amoeba or spores are stored or transported at 80° C. in 10% Dimethyl sulfoxide (DMSO) or 10% glycerol, in the MS2 medium comprising the following: peptone 10 g, dextrose 10 g, Na₂HPO₄×12H₂O 1 g, KH₂PO₄ 1.5 g, MgSO₄ 0.5 g, per 1 L, 1 g yeast extract (Raper 1984). Another method of long-term storage of spores is lyophilization.

In some embodiments, the present invention also provides pharmaceutical preparations for treating microbial infections in clinical, agricultural, research and industrial applications. In certain clinical applications, these preparations comprise one of the aforementioned amoebae/slugs or spores (FIG. 3), formulated for an administration to the patient. In some embodiments amoebae, slugs or spores are incorporated into surgical sutures, bandages, dressings, or other wound coverings. In addition, in some embodiments, spores are incorporated into salves, ointments, or other topical applications.

In some embodiments, amoebae, slugs or spores are delivered by pharmaceutically acceptable carrier, that refers to any of the standard pharmaceutical carriers including, but not limited to, saline solution, water, emulsions (e.g., such as an oil/water or water/oil, emulsions), and various types of wetting agents, any and all solvents, dispersion media, coatings, sodium lauryl sulfate, isotonic and absorption delaying agents, disintigrants (e.g., potato starch or sodium starch glycolate), and the like. The compositions also can include stabilizers and preservatives. For example, of carriers, stabilizers, and adjuvants. (See e.g., Martin, Remington's Pharmaceutical Sciences, 15th Ed., Mack Publ. Co., Easton, Pa. (1975), incorporated herein by reference). Moreover, in certain embodiments, the compositions of the present invention may be inoculated for horticultural or agricultural use. Such formulations include dips, sprays, seed dressings, stem injections, sprays, and mists.

The pharmaceutical compositions of the present invention may be administered in a number of ways depending upon whether local or systemic treatment is desired and upon the area to be treated. Administration may be topical (including ophthalmic and to mucous membranes including, mouth, nostrils and vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Pharmaceutical compositions and formulations for topical administration (e.g., to tissues, wounds, organs, etc) may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may additionally contain other adjunct components conventionally found in pharmaceutical compositions. Thus, for example, the compositions may contain additional, compatible, pharmaceutically active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the active agents of the formulation.

Dosing is dependent on severity and responsiveness of the disease state or condition to be treated, with the course of treatment lasting from several days to several months, or until a cure is effected or a diminution of the disease state is achieved. In some embodiments, treatment is administered in one or more courses, where each course comprises one or more doses per day for several days (e.g., 1, 2, 3, 4, 5, 6) or weeks (e.g., 1, 2, or 3 weeks, etc.). In some embodiments, courses of treatment are administered sequentially (e.g., without a break between courses), while in other embodiments, a break of 1 or more days, weeks, or months is provided between courses. In some embodiments, treatment is provided on an ongoing or maintenance basis (e.g., multiple courses provided with or without breaks for an indefinite time period). Optimal dosing schedules can be calculated from measurements of drug accumulation in the body of the patient. The administering physician can readily determine optimum dosages, dosing methodologies and repetition rates.

### II. Uses

Embodiments of the present invention provide compositions and methods for the therapeutic, clinical, research, agricultural and industrial use of amoebae. Exemplary applications are discussed herein. Additional uses are known to one of ordinary skill in the art.

A. Clinical and Therapeutic Applications

In some embodiments, amoebae are used in the treatment of subjects (e.g., humans or non-human animals) infected with a microorganism (e.g., pathogenic bacteria). In some embodiments, amoebae are used on infected skin wounds. At sites suffering tissue damage and infection, amoebae will consume large numbers of pathogens. This feeding behavior reduces the bacterial load sufficiently for wounds and surgical closures to heal naturally, and for grafts to thrive.

Chronically infected wounds present a significant burden to the healthcare system both in terms of individual and societal costs. Two important factors hamper successful treatment of these wounds: The lack of unified criteria for employing different treatments and the lack of proven treatment regimens. Against this backdrop of variability, the idea that a critical microbial load is a principal determining factor in wound healing has fared remarkably well. Numerous studies have demonstrated that the microbial load is a reliable predictive indicator of successful treatment outcomes (Bendy et al., (1964) Antimicrob. Agents Chemother (Bethesda) 10: 147-55; Bergstrom et al., (1994) Treatment of pressure ulcers. Clinical practice guideline, No. 15; Bowler P G. (2002) Wound pathophysiology, infection and therapeutic options. Ann Med 34(6): 419-27; Krizek T J, Robson M C. (1975) Am J Surg 130(5): 579-84; Robson M C, Heggers J P. (1969) Mil Med 134(1): 19-24; Daltrey et al., (1981) J Clin Pathol 34(7): 701-5. PMCID: PMC493797; Dow G. (2001) Infection in chronic wounds. Chronic Wound Care: A Clinical Source Book for Healthcare Professionals: 343-56). These studies all discuss that 10⁵ organisms per gram of tissue is the breakpoint beyond which wounds become non-healing. The best current practices aim at keeping the localized concentration of bacteria in wounds well below this threshold, typically through the administration of systemic antibiotics and surgical debridement (Bowler P G., 2002, Ann Med 34(6): 419-27). The treatment of chronic infections of the skin often is a challenge to clinicians. Infections, burns, surgical wounds, and diabetic lesions can be refractory to current treatment regimes causing them to persist as open sores. The most common underlying reasons for this type of pathology are: antibiotic failure due to high bacterial loads, infection with multiple antibiotic-resistant pathogens, or the formation of antibiotic-impervious biofilms. Clinicians are demanding new and more effective therapies.

Recently, owing to the frequency of therapeutic failures, there has been growing interest in the development and use of topical antimicrobial agents. Biotherapeutics for disease can be found in bacteriophage, benign bacterial themselves, and leech and maggot therapies. For instance, bacteriophage therapy, as an alternative or adjunct to chemical antibiotics can be utilized. Phage therapy uses mixtures of lytic viruses to kill pathogenic bacteria (Mann N H, 2008. Res Microbiol. 159:400-405). A second strategy, bacterial interference, uses live benign bacteria to displace pathogenic organisms. Several examples of this technology are in the research stage (Huovinen P. 2001. BMJ. 323:353-354, and U.S. Pat. No. 6,991,786). The US Food and Drug Administration has approved both leeches and maggots as Class II medical devices. Leeches are used in the treatment of venous congestion (Zhang X, et al. 2008. J Hand Surg Am. 33:1597-601), and maggots are used to disinfect and debride wounds (Hunter S, et al. 2009, Adv Skin Wound Care. 22:25-27).

The use of biologics is much broader than those examples mentioned above. For example, preparations of the prokaryote Lactobacillus acidophilus for use in human therapies is known (see, e.g., U.S. Pat. Nos. 5,032,399 and 5,733,568). In addition, pharmaceutical preparations of Lactobacillus acidophilus are known (See e.g., U.S. Pat. No. 4,314,995). Additional applications of biologics in human therapy are described in U.S. Pat. No. 5,607,672 (Using recombinant Streptococcus mutans in the mouth to prevent tooth decay); U.S. Pat. No. 6,447,784 15 (Genetically modified tumor-targeted bacteria (Salmonella) with reduced virulence); U.S. Pat. No. 6,723,323 (Vibrio cholerae vaccine candidates and method of their constructing); 6,682,729 (A method for introducing and expressing genes in animal cells is disclosed comprising infecting the animal cells with live invasive bacteria); and U.S. Pat. No. 4,888,170 (relating to a vaccine for the immunization of a vertebrate, comprising: an avirulent derivative of a 20 pathogenic microbe).

In some embodiments, amoebae are utilized in the treatment of microbial infections in mucus membranes (e.g., nostrils, throat, rectum, vagina, etc.), tissues or organs (e.g., urinary tract, etc) or bodily fluids (e.g., blood).

In some embodiments, amoebae are utilized in the treatment of infection by drug or multi-drug resistant bacteria (e.g., methycillin resistant Staph aureus (MRSA) or MDR (multi-drug resistant) Acinetobacter baumannii) or dormant persister cells.

Dormant persister cells are tolerant to antibiotics and are largely responsible for recalcitrance of chronic infections. Chronic infections are often caused by pathogens that are susceptible to antibiotics, but the disease may be difficult or even impossible to eradicate with antimicrobial therapy. For many pathogens, including S. aureus, a highly significant factor of virulence steams from the fact that in addition to fast-growing cells these pathogens produces small numbers of dormant persister cells whose function is survival in adverse circumstances. Persisters are not mutants, but phenotypic variants of the wild type, and are tolerant to killing by antibiotics. The dormancy protection from antibiotics is mechanistically distinct from genetically determined MRSA. Antimicrobial therapy, however, selects for high persistence mutants, or Small Colony Variants (SCVs). SCVs have been found for many genera of bacteria, but they have been most extensively studied for staphylococci. (Proctor et al., Clin. Infect. Dis. 20, 95-102 (1995). S. aureus SCVs can also cause more aggressive infections in both humans and animals. The high rate of selection by aminoglycosides indicates that SCVs are part of the normal life cycle of staphylococci. (Massey et al., Curr. Biol. 11, 1810-1814 (2001). Massey, R. C. & Peacock, S. J. Curr. Biol. 12, R686-R687 (2002).

Experiments conducted during the course of development of embodiments of the present invention demonstrated that soil amoebae can destroy MRSA and persister cells of the pathogen.

In some other embodiments, the present methods and compositions are directed to specifically controlling (e.g., therapeutic treatments or prophylactic measures) diseases caused by the following pathogens: Bartonella henselae, Borrelia burgdorferi, Campylobacter jejuni, Campylobacter fetus, Chlamydia trachomatis, Chlamydia pneumoniae, Chylamydia psittaci, Simkania negevensis, Escherichia coli (e.g., O157:H7 and K88), Ehrlichia chafeensis, Clostridium botulinum, Clostridium perfringens, Clostridium tetani, Enterococcus faecalis, Haemophilus influenzae, Haemophilus ducreyi, Coccidioides immitis, Bordetella pertussis, Coxiella burnetii, Ureaplasma urealyticum, Mycoplasma genitalium, Trichomatis vaginalis, Helicobacter pylori, Helicobacter hepaticus, Legionella pneumophila, Mycobacterium tuberculosis, Mycobacterium bovis, Mycobacterium africanum, Mycobacterium leprae, Mycobacterium asiaticum, Mycobacterium avium, Mycobacterium celatum, Mycobacterium celonae, Mycobacterium fortuitum, Mycobacterium genavense, Mycobacterium haemophilum, Mycobacterium intracellulare, Mycobacterium kansasii, Mycobacterium malmoense, Mycobacterium marinum, Mycobacterium scrofulaceum, Mycobacterium simiae, Mycobacterium szulgai, Mycobacterium ulcerans, Mycobacterium xenopi, Corynebacterium diptheriae, Rhodococcus equi, Rickettsia aeschlimannii, Rickettsia africae, Rickettsia conorii, Arcanobacterium haemolyticum, Bacillus anthracis, Bacillus cereus, Lysteria monocytogenes, Yersinia pestis, Yersinia enterocolitica, Shigella dysenteriae, Neisseria meningitides, Neisseria gonorrhoeae, Streptococcus bovis, Streptococcus hemolyticus, Streptococcus mutans, Streptococcus pyogenes, Streptococcus pneumoniae, Staphylococcus aureus, Staphylococcus epidermidis, Staphylococcus pneumoniae, Staphylococcus saprophyticus, Vibrio cholerae, Vibrio parahaemolyticus, Salmonella typhi, Salmonella paratyphi, Salmonella enteritidis, Klebsiella pneumoniae, Klebsiella oxytoca, Erwinia amylovora, Pseudomonas aeruginosa and Treponema pallidum.

B. Surfaces

In some embodiments, compositions of the present invention are used to treat surfaces. Surfaces that can be treated by the methods and compositions of the present invention include but are not limited to, surfaces of a medical device (e.g., a catheter, implants, stents, etc.), a wound care device, a body cavity device, a human body, an animal body, a food preparation surface, an industrial surface, a personal protection device, a birth control device, and a drug delivery device. Surfaces include but are not limited to silicon, plastic (e.g., polycarbonate), glass, polymer, ceramic, skin, tissue, nitrocellulose, hydrogel, paper, polypropylene, cloth, cotton, wool, wood, brick, leather, vinyl, polystyrene, nylon, polyacrylamide, optical fiber, natural fibers, nylon, metal, rubber, soil and composites thereof. In some embodiments, the treating destroys growing, nongrowing, or dormant microbial pathogens.

C. Agricultural Uses

In some embodiments, amoebae are used in the treatment of microbial infections of agricultural and industrial plants. For example, in experiments conducted during the course of developments of embodiments of the present invention amoebae were shown to be effective against virulent strains of Erwinia amylovora (88, 85.1 and A97.1) a causative agent of Fire blight in fruit crops. In addition, Burkholderia cepacia is a bacterium which produces economic losses to onion crops (Burkholder 1950. Phytopathology 40:115-118).

D. Biofilms

In some embodiments, the methods and compositions of the present invention target bacteria present as a biofilm. Biofilms are assemblages of microorganisms attached to natural or man-made surfaces (Costerton et al., Scientific American. 1978; 238(1):86-95). Structural heterogeneity, genetic diversity, and an extracellular matrix of polymeric substances characterize these complex and interactive communities (Hall-Stoodley et al., Nature reviews Microbiology. 2004; 2(2):95-108; Fux et al., Expert review of anti-infective therapy. 2003; 1(4):667-83; Richards et al., Chembiochem: a European journal of chemical biology. 2009; 10(14):2287-94; Burmolle et al., FEMS immunology and medical microbiology. 2010; 59(3):324-36). Biofilms occur on natural body surfaces, wounds, and medical devices (Donlan R M. Emerging infectious diseases. 2002; 8(9):881-90) and the bacteria living in biofilms are innately protected against antibiotics and disinfectants (Donlan R M, Costerton J W. Clinical microbiology reviews. 2002; 15(2):167-93) as well as the body's phagocytic host defenses (Bjarnsholt et al., Microbiology. 2005; 151(Pt 2):373-83; Walker et al., Infection and immunity. 2005; 73(6):3693-701; Mittal et al., Comp Immunol Microbiol Infect Dis. 2006; 29(1):12-26; Jesaitis et al., Journal of immunology. 2003; 171(8):4329-39; Thurlow et al., Journal of immunology. 2011; 186(11):6585-96; Kjelleberg S., Trends in Microbiology (2005) 13(7), Off the hook—how bacteria survive protozoan grazing). These features, combined with antibiotic resistance genes, cause some bacterial infections to be extremely difficult or impossible to eradicate (Richards J J, Melander C. Chembiochem: a European journal of chemical biology. 2009; 10(14):2287-94). Agriculture and animal husbandry are also confronted by serious challenges posed by biofilms (reviewed in Ramey et al., Current opinion in microbiology. 2004; 7(6):602-9; Danhorn et al., Annual review of microbiology. 2007; 61:401-22) and yet the use of antibiotics in these areas has become increasingly controversial and is drawing close scrutiny worldwide from regulators and the public (Salyers A A, and McManus, P. S., Possible impact on antibiotic resistance in human pathogens due to agricultural use of antibiotics. Andersson DHaDI, editor. Taylor & Francis, London 2001; Kieny M-P. The evolving threat of antimicrobial resistance-Options for action. Sir L Donaldson DHaDP, editor: World Health Organization; 2012). Bio-fouling presents another biofilm-induced problem, which is felt across numerous industries including food processing, utilities, and maritime transportation. As the underlying agent, biofilm costs are directly related to decreases in industrial production efficiency through energy losses, physical deterioration, and chemical interference (Hall-Stoodley et al., Nature reviews Microbiology. 2004; 2(2):95-108.). For decades, chemicals with broad-based toxicity sufficient to destroy biofilms were seen as highly desirable. However, the ill-managed application of these products has resulted in the contamination of soils, lakes, and rivers.

Biofilm accumulation on teeth and gums, urinary and intestinal tracts, and implanted medical devices such as catheters and prostheses frequently lead to infections (Characklis W G. Biofilm processes. In: Characklis W G and Marshall K C eds. New York: John Wiley & Sons, 1990:195-231; Costerton et al., Annu Rev Microbiol 1995; 49:711-45).

Biofilm formation is also thought to play a central role in a variety of systems related to human health and healthcare delivery. For example, biofilm formation has been implicated in dental carry formation, gingivitis, otitis, endocarditis, infections of medical implants such as catheters, infections accompanying cystic fibrosis, and urinary tract infections (Marsh P D (2006). BMC Oral Health 6(Suppl 1):14; Costerton J W. (1999) Science 284:1318-1322). Furthermore, biofilms can cause clouding of contact lenses, contamination of pharmaceutical and cosmetic products, and biofouling of dental units water lines and dialysis machines (Imamura Y. (2008) Antimicrob Agents Chemother 52(1): 171-182; Fischer S. (2012) GMS Krankenhhyg Interdiszip 7(1): Doc08; Uppuluri P. (2010) PLOS Pathog 6(3) e1000828).

Biofilm formation is a serious concern in the food processing industry because of the potential for contamination of food products, leading to decreased food product quality and safety (Kumar C G and Anand S K, Int J Food Microbiol 1998; 42:9-27; Wong, J Dairy Sci 1998; 81:2765-70; Zottola and Sasahara, Int J Food Microbiol 1994; 23:125-48). The surfaces of equipment used for food handling or processing are recognized as major sources of microbial contamination. (Dunsmore et al., J Food Prot 1981; 44:220-40; Flint et al., Biofouling 1997; 11:81-97; Grau, In: Smulders F J M ed. Amsterdam: Elsevier, 1987:221-234; Thomas et al., In: Smulders F J M ed. Amsterdam: Elsevier, 1987:163-180).

It has been shown that even with routine cleaning and sanitizing procedures consistent with good manufacturing practices, bacteria can remain on equipment, food and non-food contact surfaces and can develop into biofilms. In addition, L. monocytogenes attached to surfaces such as stainless steel and rubber, materials commonly used in food processing environments, can survive for prolonged periods (Helke and Wong, J Food Prot 1994; 57:963-8). This would partially explain their ability to persist in the processing plant. Common sources of L. monocytogenes in processing facilities include equipment, conveyors, product contact surfaces, hand tools, cleaning utensils, floors, drains, walls, and condensate (Tomkin et al., Dairy, Food Environ Sanit 1999; 19:551-62; Welbourn and Williams, Dairy, Food Environ Sanit 1999; 19:399-401).

Other areas in which biofilms lead to economic loss include but are not limited to citrus canker, Pierce's disease of grapes, bacterial spot of plants such as peppers and tomatoes, air handling and water handling systems, water cooling systems at nuclear plants, biofouling of paper mill manufacturing equipment, and biofouling of oil and gas piplines. (Andersen P. (2007) FEMS Microbiology Letters 274(2) 210-217; Hugenholtz P. (1995) Letters in Applied Microbiology 21(1) 41-46; Wolfram J H. (1997) Microbial Degradation Processes 11, 139-147; Lindberg L E. (2001) Appl Microbiol Biotechnol 55(5) 638-43; Neria-Gonzalez I. (2006) Anaerobe 12(3) 122-33)

Bacterial genera containing species capable of forming biofilms include but are not limited to the following: Staphlococcus, Enterococcus, Pseudomonas, Haemophilus, Escherichia, Burkholderia, Streptococcus, Legionella, Fusarium, Erwinia, Klebsiella, Candida, Listeria, Proteus, Citrobacter, Enterobacter, Halanaerobium, Desulfovibrio, and Desulfonatronovibrio (U.S. Pat. No. 7,485,324; Lewis K. (2001) Antimicrob Agents Chemother 45(4) 999-1007; Imamura Y. (2008) Antimicrob Agents Chemother 52(1): 171-182; Tomkin et al., Dairy, Food Environ Sanit 1999; 19:551-62; Wasfi R. (2012) Indian Journal of Meical Microbiology 30(1) 76-80; Lindberg L E. (2001) Appl Microbiol Biotechnol 55(5) 638-43; Neria-Gonzalez I. (2006) Anaerobe 12(3) 122-33.)

An important mortality factor in the control of bacterial populations is the uptake and killing of bacteria by phagocytic eukaryotic cells (See e.g., Matz, Biofilms and Predations, 194-213 in The Biofilm Mode of Life: Mechanisms and Adaptations, Horizon Bioscience Editor: Staffan Kjelleberg and Michael Givskov, June 2007; herein incorporated by reference in its entirety). Accordingly, embodiments of the present invention provide compositions and methods for the use of amoebae in the killing of bacteria present in biofilms.

E. Combination and Co-Therapy

In some embodiments, compositions for use in killing microorganisms utilize two or more distinct species of amoebae. Some species of amoebae use different chemoattractants while other species use the same chemoattractants. For example, for D. mucoroides it is cyclic AMP, while that of P. violaceum is a dipeptide called glorin. This means that when the aggregation centers are first formed, each species is producing its own attractant and will attract only the amoebae that respond to it; they will have no interest in the attractant of the other species and therefore no possibility of commingling.

In another case, Raper and Thom chose two species that had the same chemoattractant, which is cyclic AMP (Raper, K. B., and C. Thorn (1941) Am. J. Botany 28: 69-78). Strains were D. mucoroides with white sori and D. purpureum with purple sori. The authors found that these two species co-aggregated into common centers, but there was a surprising sequel. Fruiting bodies arose from the same mound and their sorocarps were either white or purple: the amoebae had separated into two groups in the mound, and the resulting fruiting bodies were pure and all their amoebae were of either one species or the other.

Yet in another case, H. Hagiwara (Hagiwara, H. (1989) The taxonomic study of Japanese Dictyostelid cellular slime molds. Tokyo: National Science Museum Press) discovered a strain of P. pallidum that produces a substance that destroys many other strains of P. pallidum as well as a common wild-type strain D. discoideum. They do so by secreting a lethal molecule that devastates the amoebae of the susceptible victim.

Thus, in some embodiments, two or more compatible species are utilized in a composition. Such combinations are contemplated to find particular use in the killing of drug resistant microorganisms and mixed populations of microorganisms.

In some embodiments, one or more amoebae are administered in combination with known anti-microbial agents. There are an enormous amount of antimicrobial agents currently available for use in treating bacterial and fungal. For a comprehensive treatise on the general classes of such drugs and their mechanisms of action, the skilled artisan is referred to Goodman & Gilman's “The Pharmacological Basis of Therapeutics” Eds. Hardman et al., 9th Edition, Pub. McGraw Hill, chapters 43 through 50, 1996, (herein incorporated by reference in its entirety). Generally, these agents include agents that inhibit cell wall synthesis (e.g., penicillins, cephalosporins, cycloserine, vancomycin, bacitracin); and the imidazole antifungal agents (e.g., miconazole, ketoconazole and clotrimazole); agents that act directly to disrupt the cell membrane of the microorganism (e.g., detergents such as polmyxin and colistimethate and the antifungals nystatin and amphotericin B); agents that affect the ribosomal subunits to inhibit protein synthesis (e.g., chloramphenicol, the tetracyclines, erthromycin and clindamycin); agents that alter protein synthesis and lead to cell death (e.g., aminoglycosides); agents that affect nucleic acid metabolism (e.g., the rifamycins and the quinolones); and antimetabolites (e.g., trimethoprim and sulfonamides). Various combinations of antimicrobials may be employed.

### EXPERIMENTAL

The following examples are provided in order to demonstrate and further illustrate certain preferred embodiments and aspects of the present invention and are not to be construed as limiting the scope thereof.

### Example 1

### Methods

The idea of amoebic therapy is unorthodox. Various publications report the typical growth conditions for amoebae isolates: solid media composed of natural product extracts, soil bacteria (food source), and 22° C. incubation at atmospheric oxygen (Raper K B, Rahn A W. (1984) The Dictyostelids). In some embodiments, wounded tissue is at an elevated temperature (Ring E F J. 1986). Bioeng Skin 2(1): 15-30; Forage A V. (1964) Br J Plast Surg 17: 60-1; McGuiness W, Vella E, Harrison D. (2004) J Wound Care 13(9): 383-5) or hypoxic (Mathieu D. (2006) Int J Low Extrem Wounds 5(4): 233-5) or it may contain serum components that neutralize the amoebae (Ferrante A. (1991) Parasite Immunol 13(1): 31-47). Wound conditions are somewhat ill-defined and most likely vary with the type of wound. In such embodiments, culture conditions and choice of amoebae are optimized to match the intended use. In early studies that investigated the ability of D. discoideum to consume different bacteria, only one out of the hundred species of bacteria tested was not consumed. Instead, that species killed the amoebae (Raper et al. (1939) J Bacteriol 38(4): 431-45). More recent studies of bacterial pathogenesis that were also limited to D. discoideum found a few other species of bacteria that act in this same manner (e.g. species of Legionella, Pseudomonas and Mycobacterium) (Matz (2005) Trends Microbiol 13(7): 302-7).

Prior to the experiments described below, there appears to have been very little interest in examining the factors that affect the vegetative growth of soil-borne amoebae. As an endpoint, most growth studies enumerated sorocarps, the product of a completed asexual cycle (Raper K B. (1956) Mycologia 48(2):160-205; Raper (1984) The Dictyostelids; Singh B N. (1947) J Gen Microbiol 1(1): 11-21). However, amoebic therapy is primarily concerned with the viability and feeding behavior of vegetative amoebae; their social behaviors are not required for therapeutic applications.

In initial experiments, it was determined if randomly selected strains Dictyostelids, D. dictyostelium being a minority of the isolates, from the K. Raper Archive (WS 57.7; WS 645; WS 255; WS 142; WS 309; FR14; Salvador; Tu4b; X3; WS 321.5; WS 321.7; WS 371A; Turkey 27) can consume well-studied medical and agricultural pathogens. All amoebae described in the experimental section are available from Dr. Marcin Filutowicz, Department of Bacteriology, University of Wisconsin, Madison. These studies were done under several proxy conditions for wound infection (e.g. temperature, light, pH, presence of serum, oxygen concentration) agricultural virulence (temperature, oxygen etc.), and compatibility with materials used to produce medical devices (e.g. polycarbonate). The bacterial species employed in the work were all clinical/field isolates of common pathogens (e.g., Klebsiella pneumoniae, K. oxytoca, Staphylocococcus aureus (including its MRSA-USA300 derivative), Erwinia amylowora, Pseudomonas syringie, Listeria monocytogenes). The screens identified a group of amoebae that consume all tested pathogens under the proxy conditions, thus making them suitable for biotherapeutic or surface decontamination uses. Preferred candidates are those that most vigorously devour the test bacteria—in other words, the amoebae producing the largest clearing zones on lawn of specific bacteria, or rapidly consuming biofilm-encased bacteria on surfaces. For example, to meet the therapeutic criteria, the amoebae should have the ability to: consume the test bacteria, whether growing or not growing, grow/divide at the elevated temperature, grow/divide under hypoxic conditions, and grow/divide in the presence of sera. Additionally, to meet the surface-decontamination criteria, the amoebae should have the ability to consume the test bacteria, which are biofilm-encased (e.g. on surfaces made of glass or polycarbonate)

Petri-Dish Assay for the Efficiency of Amoebic Feeding and Development:

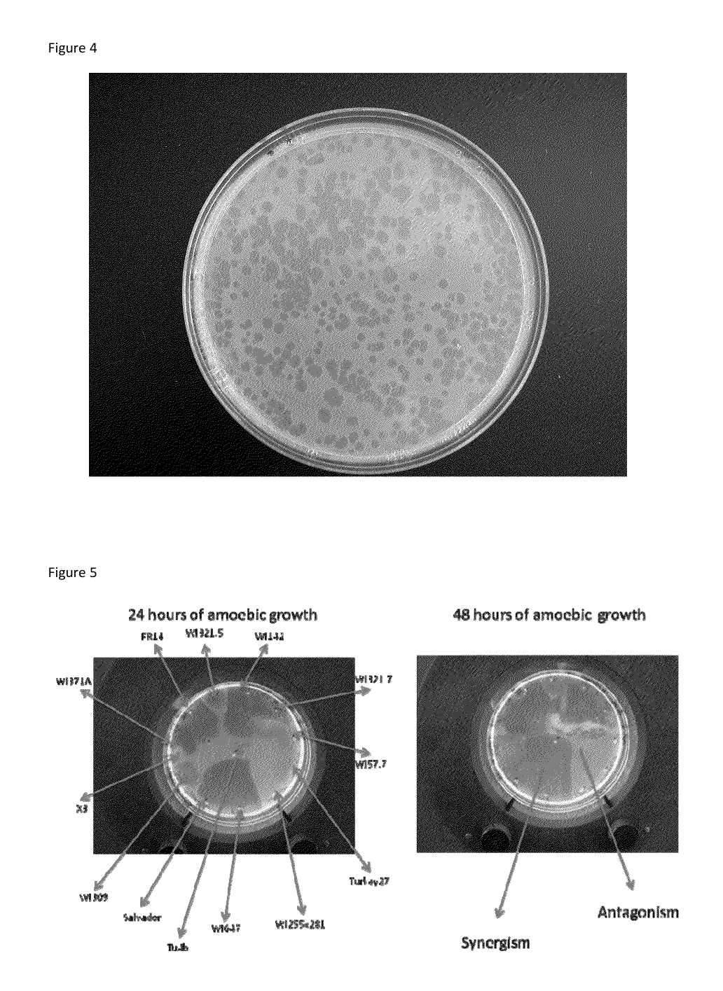

To identify putative therapeutic amoebae, a Petri-dish growth assay was used. This assay resembles a bacteriophage growth and enumeration assay in which mixtures of phage and susceptible bacteria are co-cultured as monolayers on solid medium. Each bacteriophage-infected cell gives rise to a clear plaque within the lawn of bacteria. As seen in FIGS. 1 and 4, similar zones of clearing are observed when WS-647 amoebae are co-cultured with bacteria. In this work, mixtures of amoebae (or spores) and test pathogens are co-cultured on solid medium (bacterial lawns) or resuspended in semi-solid agar. If the amoebae digest the pathogens, plaques or clearing zones appear on the lawn as the amoebae consume the bacteria. The size of the clear zones is recorded as a measure of the rate of amoebic feeding (compare FIG. 1 and FIG. 4).

Bacterial pathogens were grown in SM2 broth (e.g., E. amylovora) or other growth supporting medium (e.g. Tryptic Soy Broth, S. aureus) and organisms were removed from the medium by centrifugation. The bacterial cell pellets from these cultures were then plated on the surface of solid medium or resuspended in pre-warmed semi-solid medium (containing 0.6-1% agar). Then spores or amoebae themselves were spotted on lawns of test bacteria and after incubation (e.g., at a desired temperature, presence/absence of serum) photographed without or with magnification, and with or without a light diffuser, using various types of photographic equipment (indicated in the Fig. legends and below). In some experiments microaerophylic conditions were created using microbiological tubes containing growth supporting microbiological media. In other experiments involving agricultural pathogens (e.g. E. amylovora), pathogens were grown in SM2 semi solid medium, which was impregnated with a pear slice or supplemented with a homogenized pear according to the established protocols (Vanneste, et al. 1990 J. Bact., 1990, p. 932-941 Vol. 172; Won-Sik Kim et al., Microbiology (2004), 150, 2707-2714).

Assay for Determining Efficacy of Biofilm Feeding by Amoeba Strains:

The ability of dictyostelids to feed on bacterial biofilms (as defined by Costerton J W, Geesey G G, Cheng K J. 1978. How bacteria stick. Sci. Am. 238:86-95.) has not previously been tested. Klebsiella pneumoniae, Klebsiella oxytoca, MRSA USA300 and Erwinia amylovora bacteria with well-characterized biofilm-forming properties were used to determine if dictyostelids are able to consume biofilms. Here, it was demonstrated that several such strains can prey on biofilm-encased cells of these bacterial species. When the bacterial prey becomes limiting, most dictyostelids examined can aggregate and undergo a complete developmental cycle, although the composition of the underlying surface (glass vs. polycarbonate) and the number of bacteria available as food source can influence the outcome.

To identify strains of amoeba capable of feeding on biofilms, a time-lapse feeding assay was performed. Biofilms of selected bacteria were grown on glass coverslips, according to methods described in (Brock T D. Science. 1967; 158(3804); Walker J N, Horswill A R. Front Cell Infect Microbiol. 2012; 2:39). The microscopy experiments are best suited for generating qualitative data on biofilm destruction rather than quantitative data. To perform quantitative analyses of biofilm destruction, series of experiments in which prey and predator assemblies can be removed from the medium at any point and quantified were performed. This was achieved by forming biofilms atop microporous polycarbonate filters that can be laid on agar surfaces during incubation periods and removed for analysis. The preformed K. oxytoca, MRSA USA300, E. amylovora-biofilms were inoculated with the spores of the dictyostelid predators. The procedure is derived from the methods of Anderl and colleagues, who used polycarbonate filter membranes to support the growth of K. pneumoniae biofilms (Anderl et al., Antimicrob Agents Chemother. 2000; 44(7):1818-24). Data from a variety of Dictyostelid strains and bacterial species are presented below.

### Example 2

### Pair-wise employment of more than one strain of amoebae

This example describes the used of two or more types of amoebae to assure that the treated surface/tissue becomes microorganism-free (other than the presence of amoebae themselves, or their various social stages of development; e.g. slugs or sorocarps). Relevant to that, intra- and inter-species chemical communications among amoebae are considered and tested to choose right (compatible) partners. As shown in FIG. 5, some amoebae isolates (e.g., Salvador, and WS-647 or WS-321.7 and WS-142) seem totally unaware of each other's presence as evidenced by the overlapping clearing zones they produce. Therefore, their combination is suitable for use in a biotherapeutic cocktail of amoebae. Other amoebae isolates show a very strong antagonistic behavior (e.g. WS-255 and WS-647 or WS-321.5 and either FR14 or WS142) as evidenced by the non-overlapping clearing zones they produce.

### Example 3

### Growth Temperature

In general, dictyoslelid amoebae are propagated at a temperature between 21-25° C. (Raper K B. (1951) Q Rev Biol 26(2): 169-90; Raper K B, Rahn A W. (1984) The Dictyostelids). Temperatures above 25° C. can inhibit the growth of many species of amoebae. Such species can be employed in many, perhaps all agricultural and industrial applications (e.g., against E. amylovora, P. syringiae, and L. monocytogenes). Although dermatological wounds typically have comparable surface temperatures (between 24-26° C.), they can measure 35° C. or even higher (Ring E F J. (1986) Bioeng Skin 2(1): 15-30; Forage A V. (1964) Br J Plast Surg 17: 60-1; McGuiness W, Vella E, Harrison D. (2004) J Wound Care 13(9):

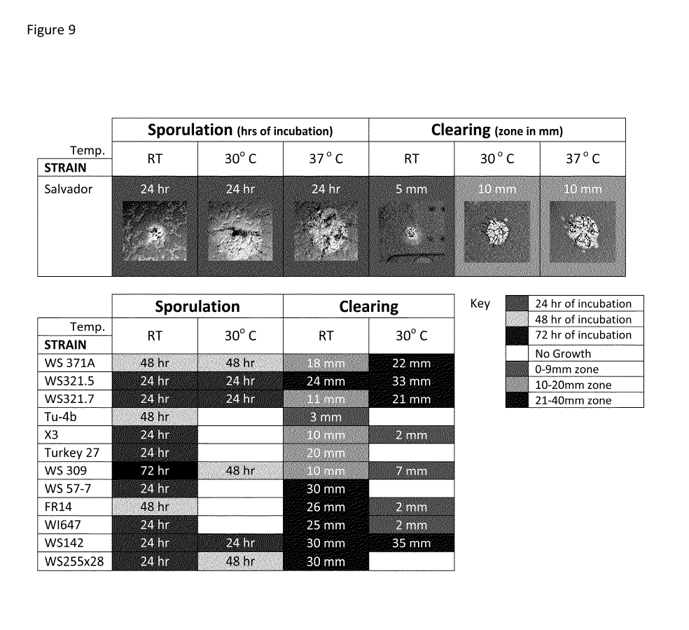

383-5). In published reports, the determination of growth temperatures relied on observing fruiting body formation, not the ability of free-living amoeba to feed on bacteria. Experiments were performed to determine if the observed temperature restriction affects bacteria-consuming amoebae or a developmental step in sorocarp formation. Klebsiella pneumoniae (10⁵ cells) was inoculated in Petri dishes using a standard overlay procedure and grown overnight to produce confluent lawns. The overlays were seeded with the indicated strains of amoebae as described in FIG. 9. Plates were incubated at various temperatures (as indicated) and data was recorded at the times shown in the key (Sporulation) and after 84 hours (Clearing). Sorocarps (spore carriers) were photographed using a camera attached to a Zeiss microscope at 8× magnification; for clearing zones, an Olympus camera without magnification was used. Images from the Salvador strain are provided to illustrate phenotypes. It was the only strain in the subset that grew at 37° C. Data on the other strains have been categorized according to the key. As amoebae feed on a bacterial lawn, they grow and multiply. Over time, a zone of clearing is formed and amoebae undergo development into sporangia. The data demonstrate variability in these phenotypes among the strains tested.

As shown in FIG. 9, amoebae were identified that can grow at temperatures of 30° C. (WS-371A, WS-321.5, WS-321.7, WS-309, WS-142, WS-255, or even 37° C. (Salvador) whereas strains of amoebae tested grew only at room temperature (Tu4b, X3, Turkey 27 WS-57.7, FR-14, WS-647). Such temperature-tolerant strains (e.g. Salvador) can prosper on surface of human/animal wounds and nonsterile nostrils or other mucosal surfaces. Therefore, such strains of amoebae are ideal for treating infected wound lesions and other mucosal surfaces.

### Example 4

### Growth in Hypoxic Conditions

In an infected wound, it is possible that amoebae will encounter hypoxic conditions because of inflammation, edema and compromised vasculature (Mathieu D. (2006) Int J Low Extrem Wounds 5(4): 233-5). Amoebae are known to grow well in the presence of oxygen and have been reported to become quiescent under anaerobic conditions (Bonner J T. The Social Amoebae The Biology of Cellular slime molds. Princeton: Princeton University Press; 2009). However, except for a single study on the development of submerged isolates of D. mucoroides, the tolerance of amoebae to anoxic environments has not been formally investigated (Sternfeld et al., (1977) Proc Natl Acad Sci USA 74(1): 268-71).

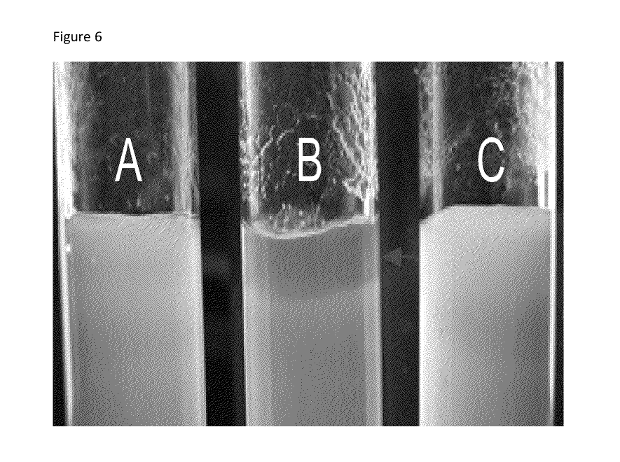

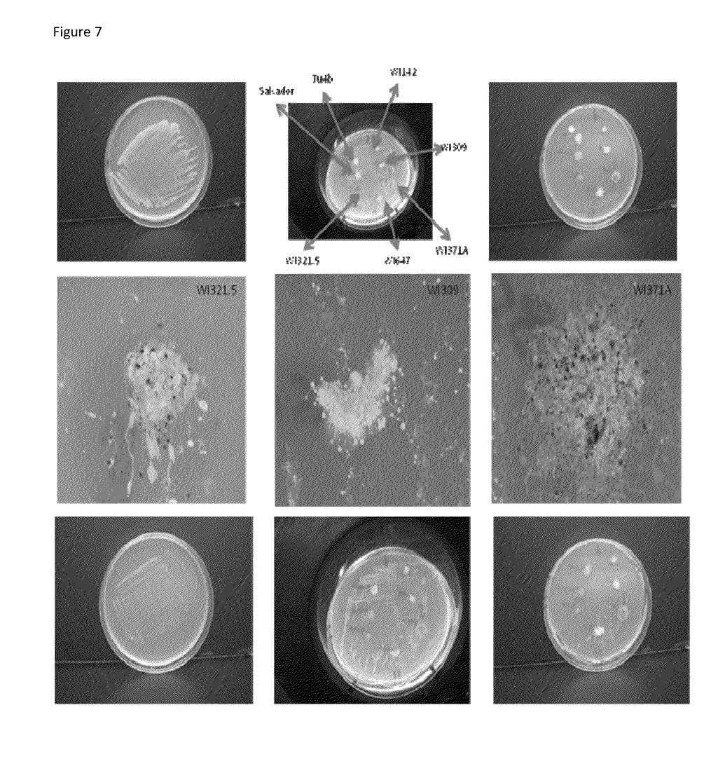

An experiment was performed that tested the ability of amoebae to penetrate throughout top agar seeded with bacteria. As shown in FIG. 6, the results demonstrate that strains differ in their oxygen requirements. WS-647 was shown to tolerate microaerophylic conditions. In majority of situations the bacteria on which the amoebae feed are embedded in biofilms, if so, oxygen levels may be reduced 1000-fold compared to atmospheric oxygen (Xu et al., (1998) Appl Environ Microbiol 64(10): 4035-9). To provide additional supportive evidence for amoebas' ability to burrow into biofilm mass, confocal laser scanning microscopy (CLSM) was used to measure the thickness of bacteria (K. oxytoca) grown using each method (glass coverslip and polycarbonate filter) for biofilm formation. The detection of bacterial cells relied on the use of fluorescent K. oxytoca, which was obtained by introducing a plasmid construct (pFL300) that encodes the red fluorescence protein (RFP) into that strain. Biofilms generated using the labeled cells were subjected to CLSM. It was found that the procedure produced multilayered bacterial biofilms on the glass surface with a thickness of 50-75 mm near the edges (FIG. 3A). CSLM was also conducted on biofilm(s) on polycarbonate filters that were inoculated with the reporter K. oxytoca, strain incubated to achieve a density of 10⁹ CFU/filter, washed extensively with saline, and photographed. The images show a mass of brightly fluorescent RFP-producing cells, which are bound to the surface despite repeated washes with saline, a criterion used by others to distinguish biofilms from planktonic cells (Merritt et al., Curr Protoc Microbiol. 2005; Chapter 1: Unit 1B).

Cells (non-fluorescent) of the dictyostelid strain WS-142 were introduced to a “coverslip biofilm” of the fluorescent derivative of K. oxytoca, strain KOF001. The interaction of predator and prey was captured using CLSM. Short videos of bottom Z-stack and top Z-stack provided additional data indicating that biofilms can form on the coverslip surface, and also allowed observation of the dynamic state of the biofilm in three dimensions. The architecture of the biofilm is comprised of a largely static central mass of multiple layers of cells. Within the biofilm mass, “pools” of free-living bacteria can be seen. Furthermore, it is evident that the extracellular matrix of KOF001 is not so rigid that it prevents dictyostelid cells of WS-142 from migrating into the biofilm in three dimensions. At several point (both positional and temporal) myxamoebae exhibit “burrowing” behavior, disappearing from view and reappearing at a nearby location. This is true for myxamoebae that are in close proximity to the glass surface (bottom Z stack) as well as those which are closer to the biofilm surface (top Z stack).

Oxygen limitations in the amoebic treatment (if needed) are addressed by the use oxygen-producing dressing on wounds and treated surfaces. This approach has been successfully employed in the therapeutic use of maggots where hypoxia is known to limit therapeutic effectiveness (Sherman R A. (1997) Plast. Reconstr. Surg. 100(2): 451-6).

### Example 5

### Growth in the Presence of Serum

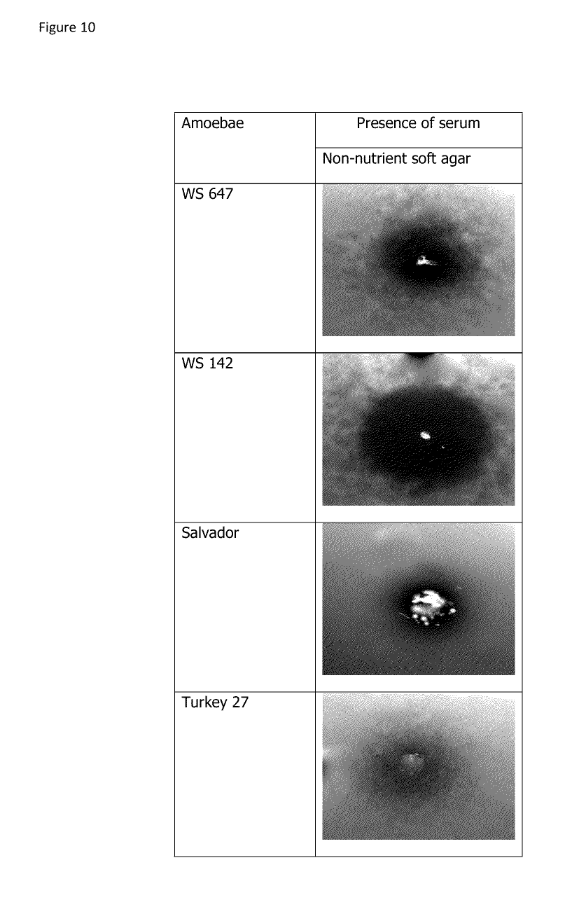

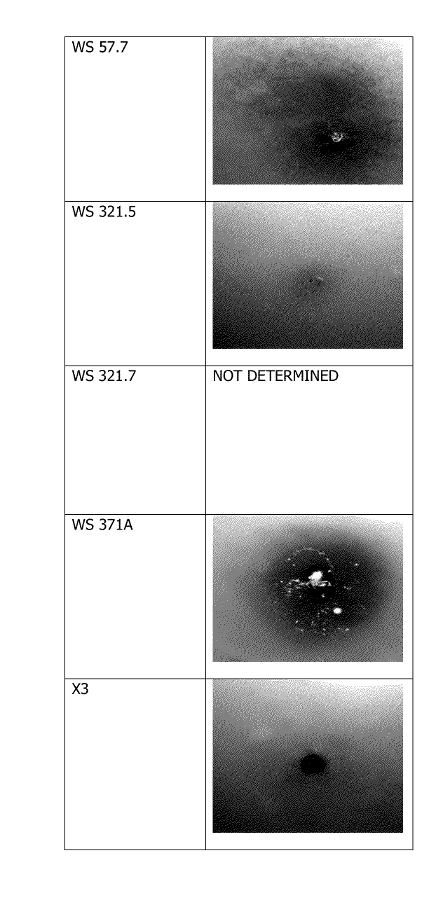

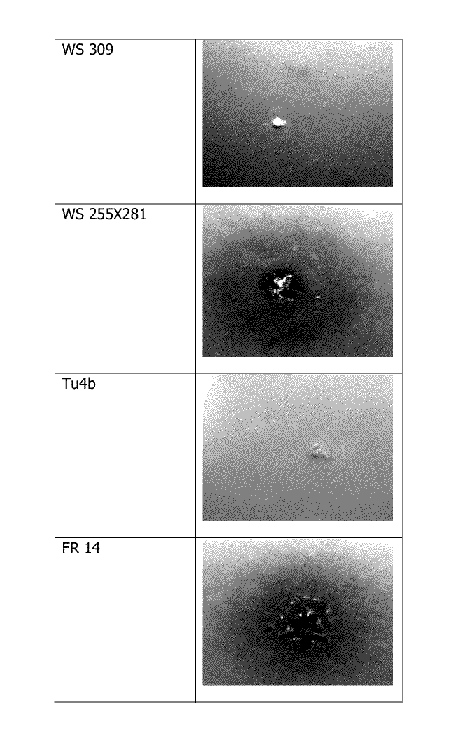

Toxic amoebae have been shown to be susceptible to serum (Cursons et al., (1980) Infect Immun 29(2): 401-7; Ferrante A. (1991) Parasite Immunol 13(1): 31-47). FIGS. 10-13 demonstrate that selected amoebae isolates can feed on several species of bacterial pathogens in the absence or presence of bovine or porcine sera. Furthermore, they can feed on bacteria re-suspended in media supporting or not supporting their growth.

FIG. 10 shows feeding of amoebae on MRSA USA300. The plates were inoculated by using the pathogen grown overnight in TSB medium. Bacteria were pelleted and resuspended in semi-solid agar containing bovine serum diluted seven-fold in semi-solid agar supplemented with 0.9% sodium chloride. Plates were incubated at 35° C. and data was recorded after 48 hours. Plates were photographed using a camera attached to an Olympus microscope at 8× magnification. Clearing zones indicate lawns of bacteria destroyed by feeding and dividing amoebae. Structures inside are showing aggregation, slugs and mature fruiting bodies of amoebae with spores. Photos were taken without a light diffuser in the microscope resulting in an enhanced contrast between clearing zones and confluent bacterial growth.

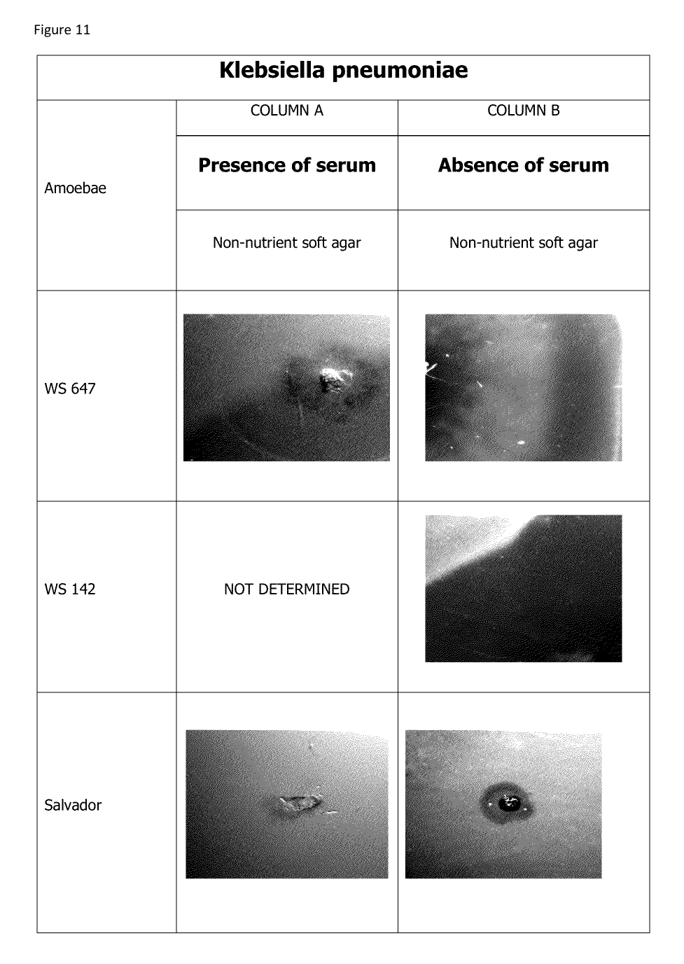

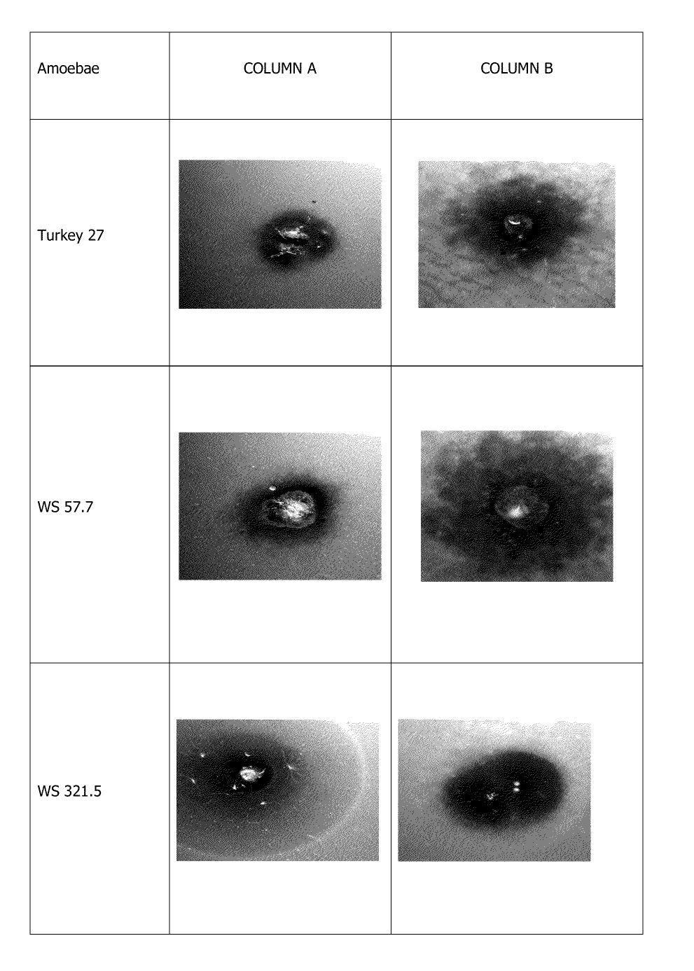

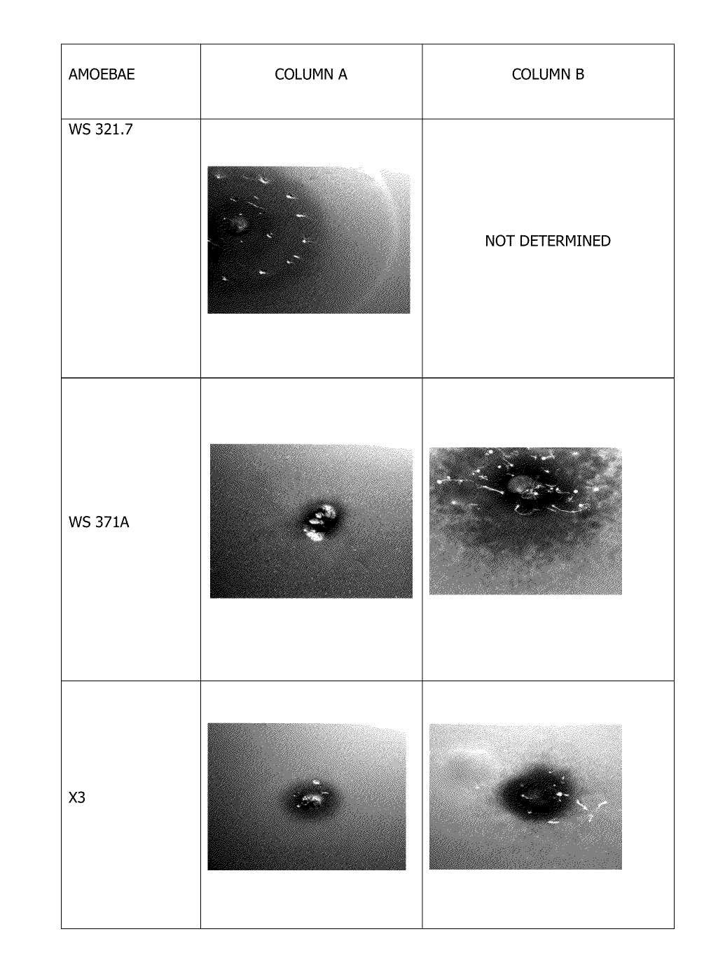

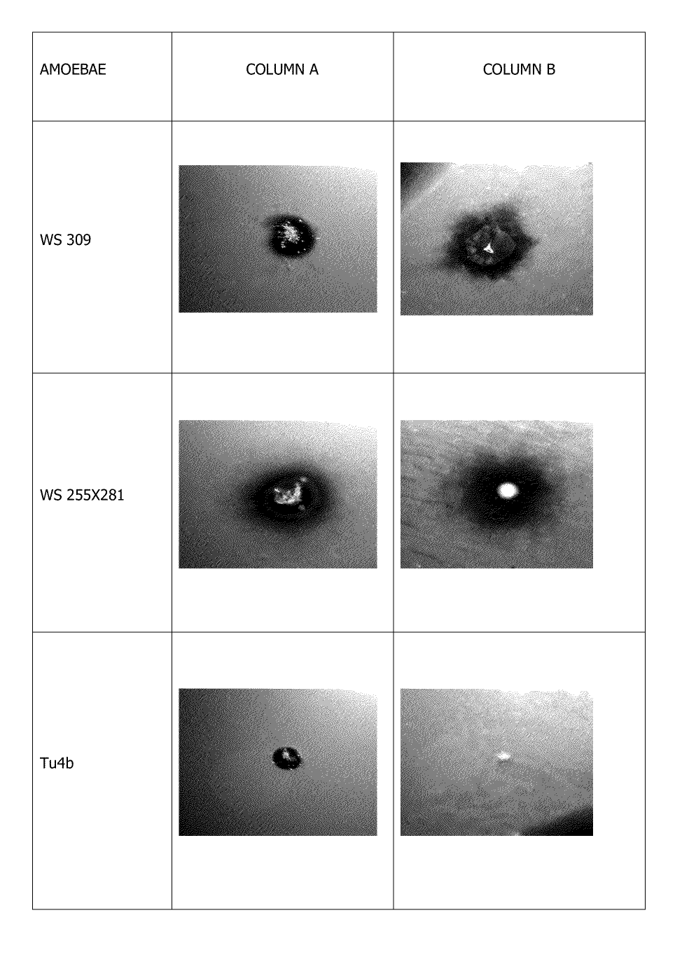

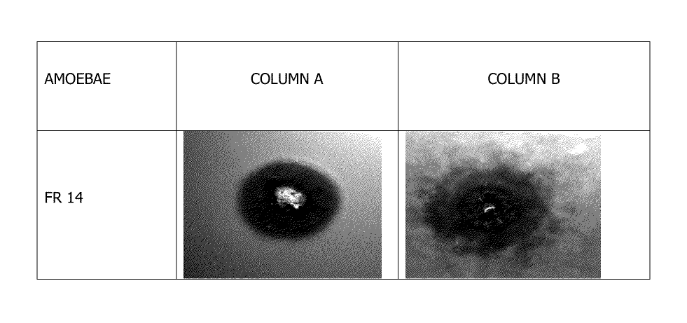

FIG. 11 shows a comparison of the feeding of amoebae on K. pneumoniae with and without serum. Column A shows zones of growth (or lack thereof) of amoebae on plates inoculated with Klebsiella pneumoniae. The plates were inoculated by using the pathogen grown overnight in SM2 medium. Bacteria were peleted and resuspended in semi-solid agar containing 0.9% sodium chloride and seven-fold diluted bovine serum (column A) or in semi-solid medium without bovine serum (Column B). Plates were incubated at room temperature and data was recorded after 48 hours (Clearing). Plates were photographed using a camera attached to an Olympus microscope at 8× magnification. Clearing zone shows feeding front of amoebae and structures inside are showing aggregation, slugs and mature fruiting bodies of amoebae with spores.

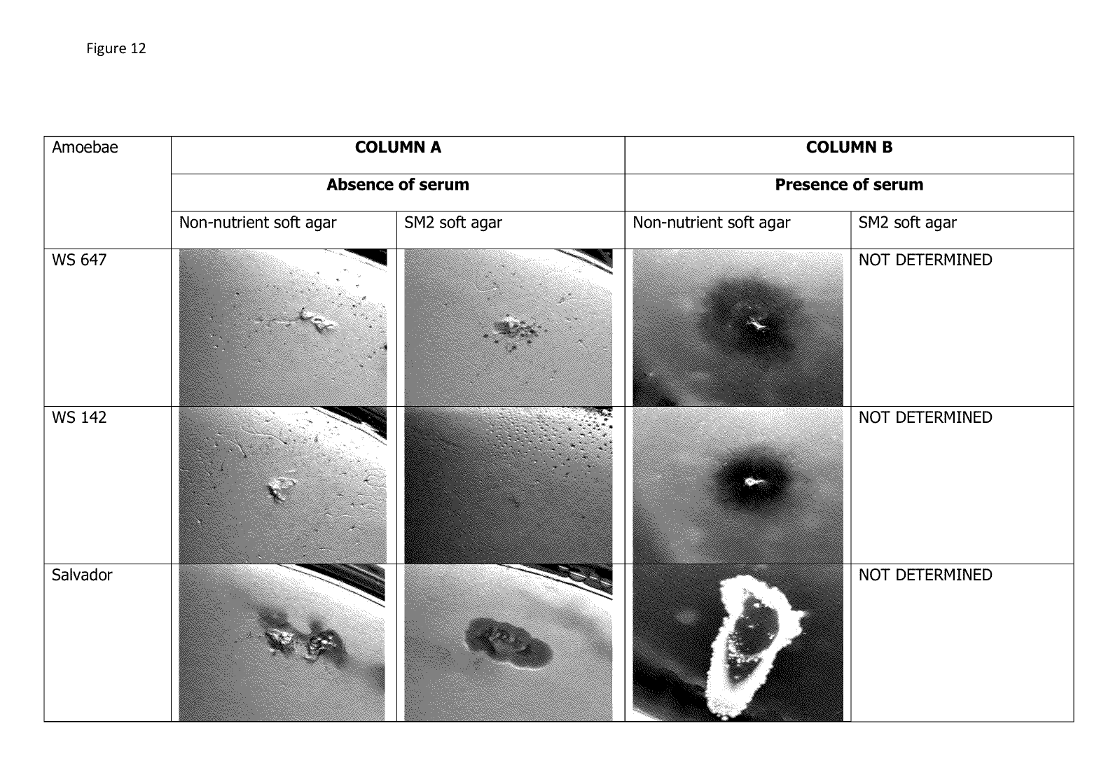

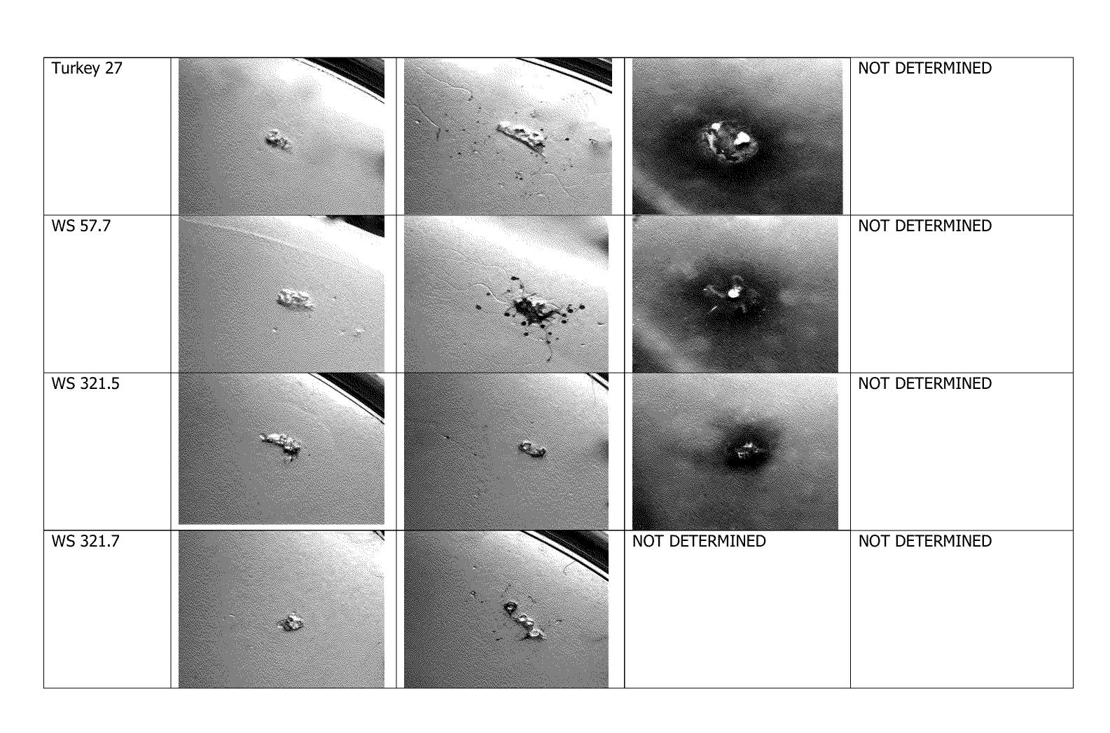

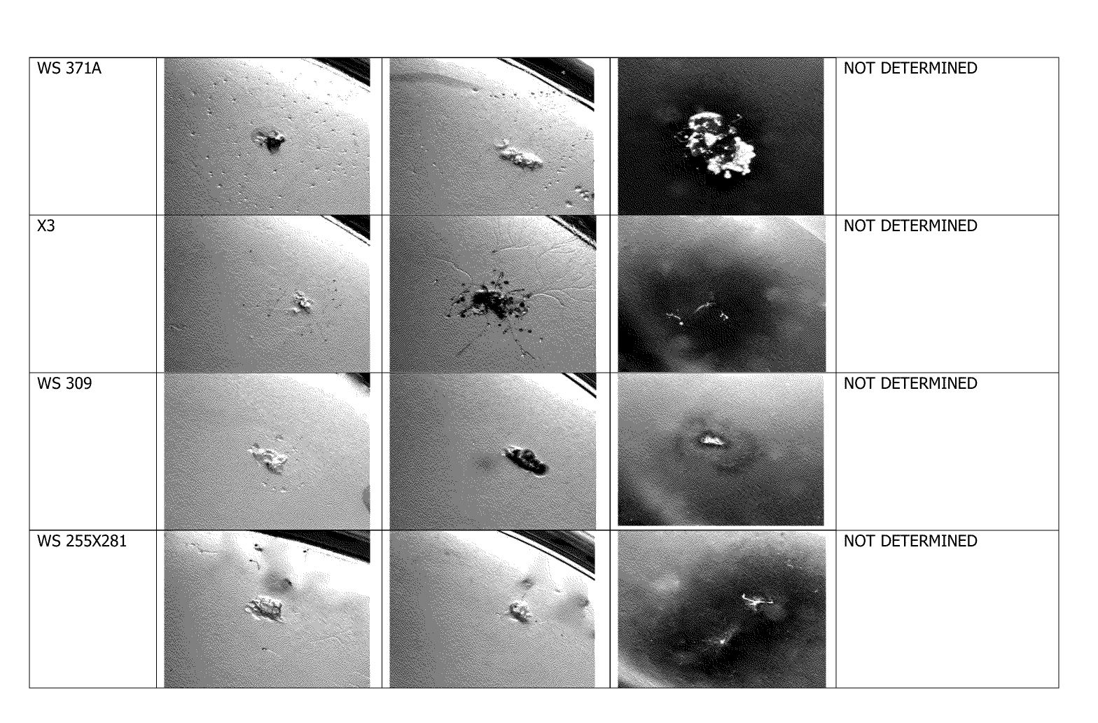

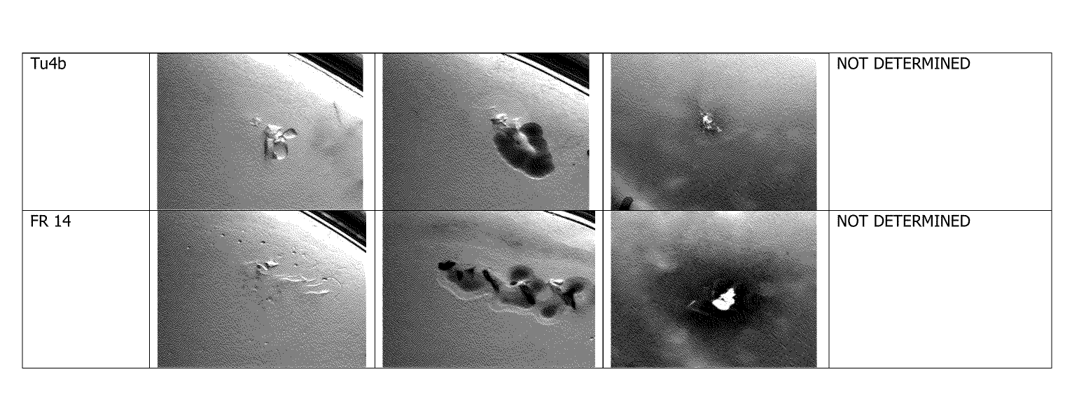

Small Colony Variants have been extensively studied for staphylococci, and it is clear they present a significant therapeutic challenge (Proctor R A. (2008) Clin Infect Dis 46(4): 584-93). The menD mutant of S. aureus that grows extremely slowly without menadione is altered in electron transport and without this compound expresses a phenotype of Small Colony Variant (von Eiff et al., (2006) J Bacteriol 188(2): 687-93; Bates et al., (2003) J Infect Dis 187(10): 1654-61). The susceptibility of this mutant to killing by amoebae was tested and it was found that all strains that kill MRSA also kill the menD mutant (without and with exogenously added menadione). FIG. 12 shows feeding of amoebae on a menD mutant of S. aureus in the presence and absence of serum. Shown are pictures of amoebae feeding on the menD mutant. The plates were made by using the pathogen grown overnight in TSB medium. Bacteria were peleted and resuspended in semi-solid agar containing 0.9% sodium chloride without bovine serum (Column A) and seven-fold diluted bovine serum (column B). Each set of the plates was supplemented with either soft agar (left) or SM2 medium (right) not supplemented with menandione (vitamin K). Plates were incubated at room temperature and data was recorded after 48 hours (Clearing). Plates were photographed using a camera attached to an Olympus microscope at 8× magnification. Clearing zone shows feeding front of amoebae and structures inside are showing aggregation, slugs and mature fruiting bodies of amoebae with spores. Photos in a column marked as (“non nutrient agar”) were taken without a light diffuser in the microscope. Therefore, contrast between clearing zones and confluent bacterial growth is enhanced in comparison to the two panels in the column A (absence of serum).

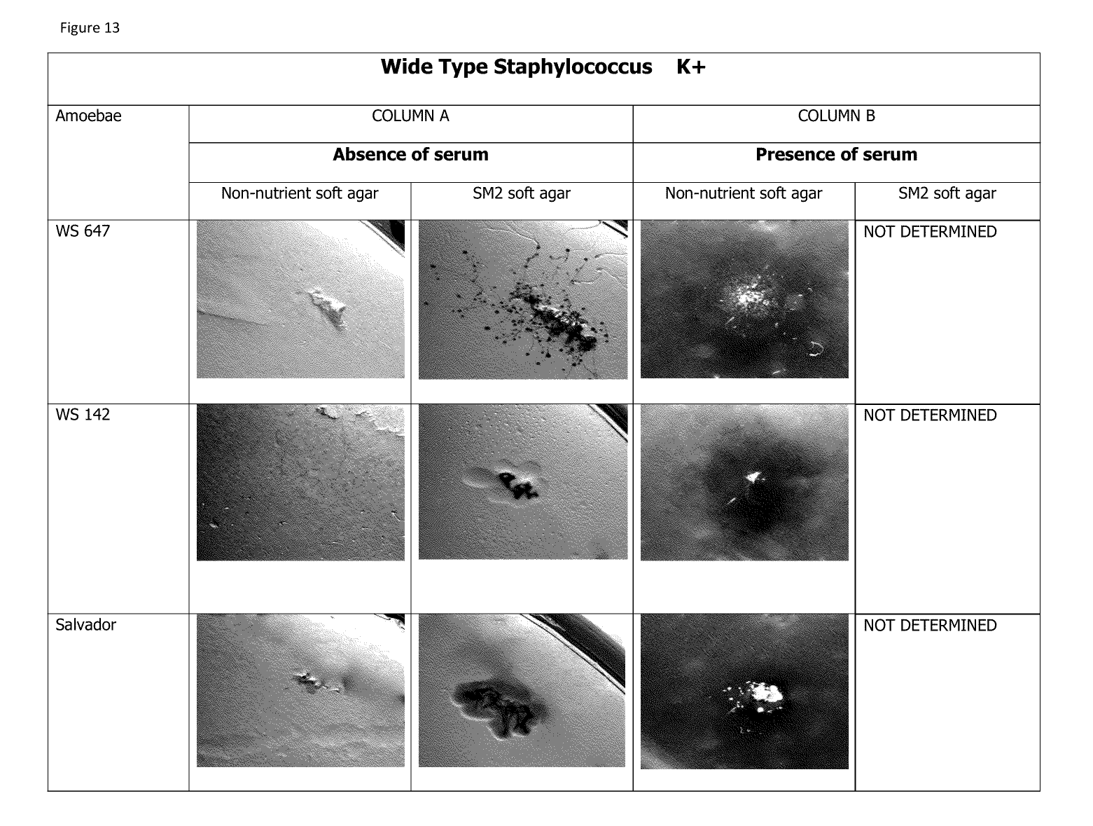

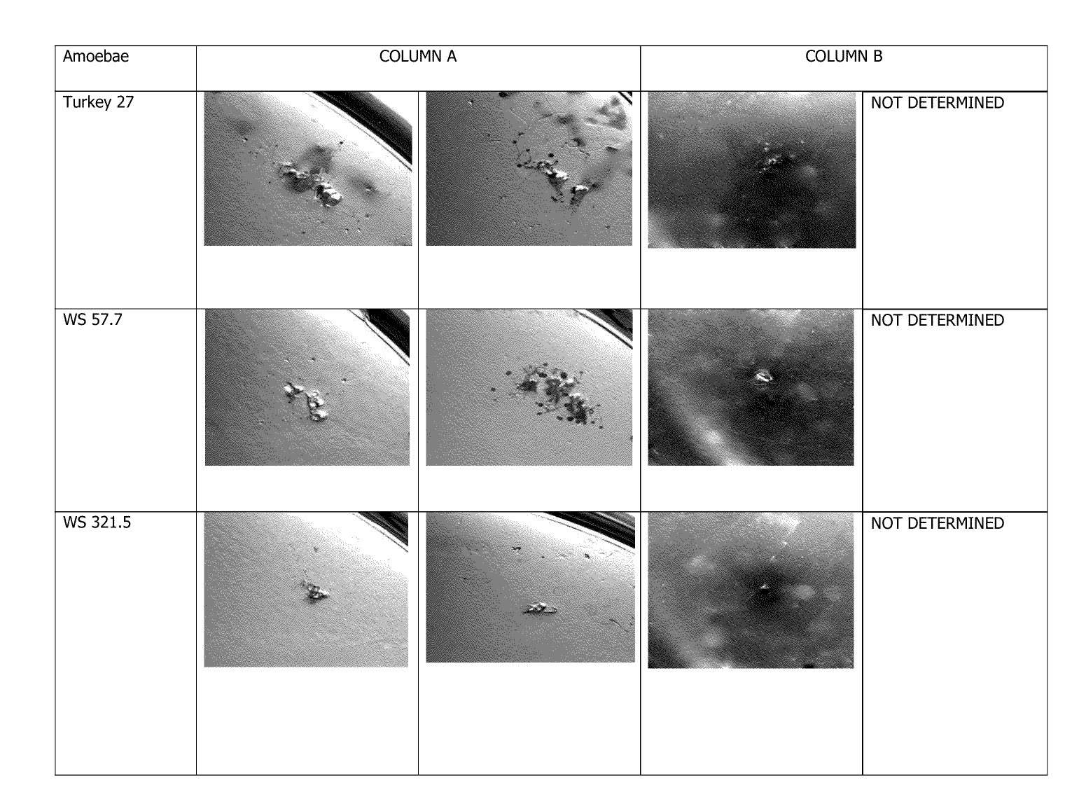

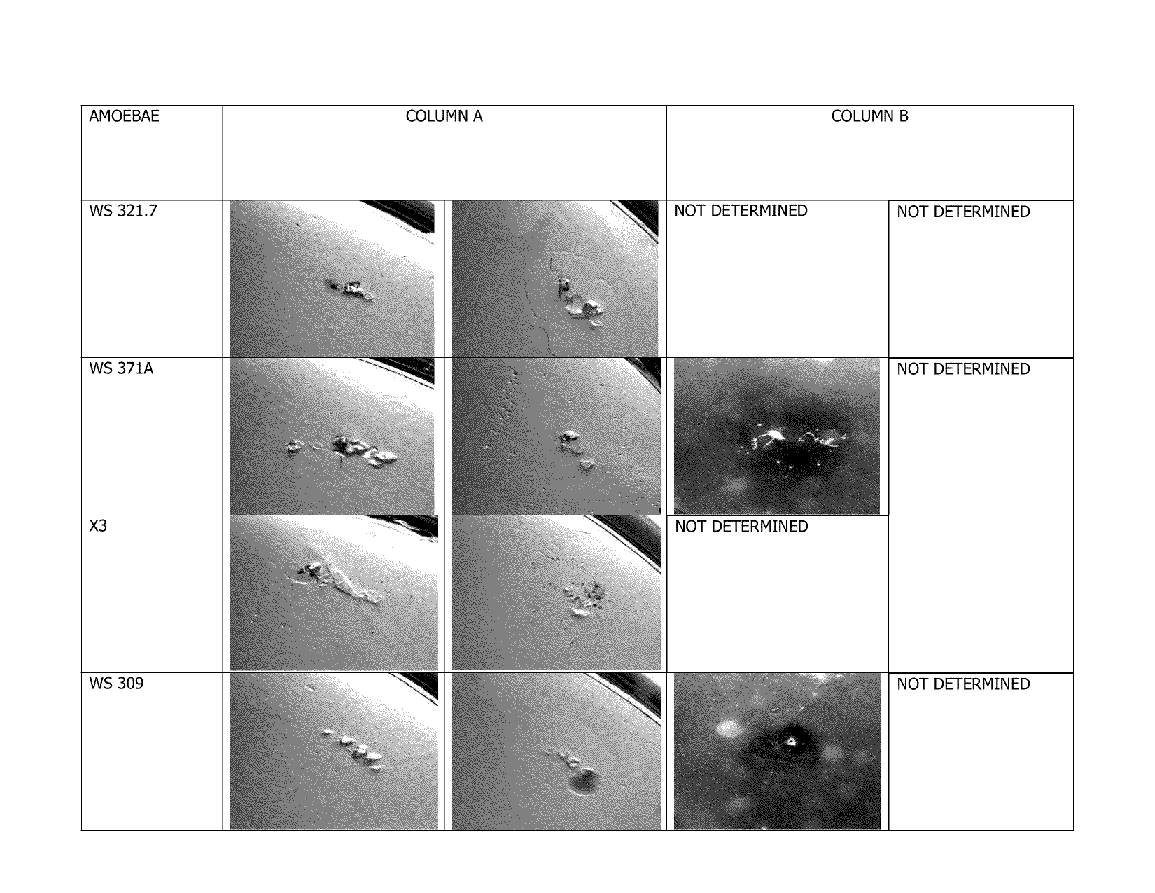

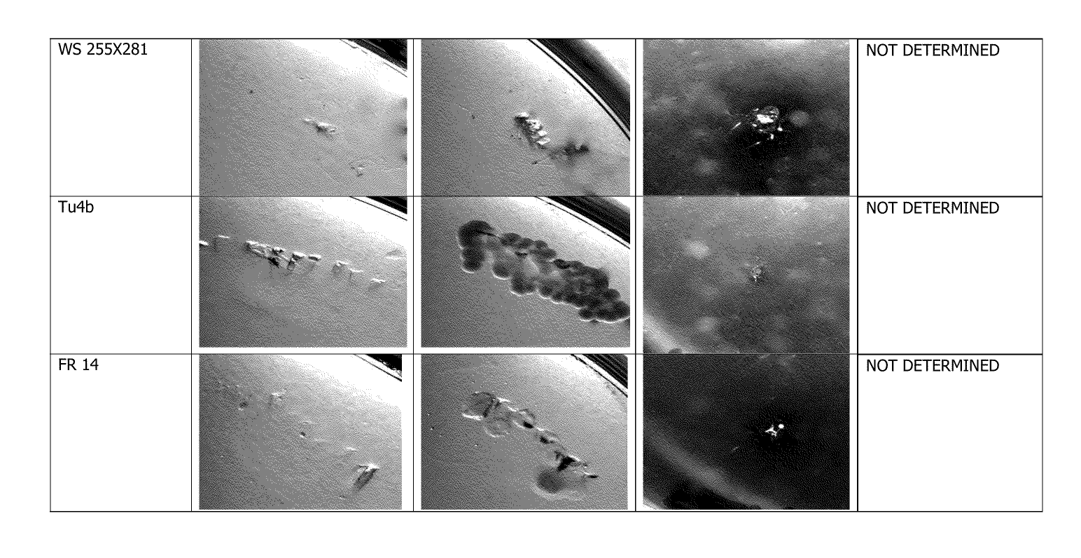

FIG. 13 shows feeding of amoebae on menD mutant (cultured in the presence of menandione and as such not expressing the mutant phenotype, therefore are designated as wild Type Staphylococcus K+) in the presence or absence of serum. Shown are pictures of amoebae feeding on the strain. The plates were made by using the pathogen grown overnight in TSB medium. Bacteria were pelleted and resuspended in semi-solid agar containing 0.9% sodium chloride without bovine serum (Column A) and seven-fold diluted bovine serum (column B). Each set of the plates was supplemented with either soft agar (left) or SM2 medium (right). Plates were incubated at room temperature and data was recorded after 48 hours (Clearing). Plates were photographed using a camera attached to an Olympus microscope at 8× magnification. Clearing zone shows feeding front of amoebae and structures inside are showing aggregation, slugs and mature fruiting bodies of amoebae with spores. Photos in a column marked as (“non nutrient agar”) were taken without a light diffuser in the microscope. Therefore, contrast between clearing zones and confluent bacterial growth is enhanced in comparison to the two panels in the column A (absence of serum).

Feeding was not affected or minimally effected by serum with the following dictyostelid strains: WS-321.7, WS-255, FR-14. Modest inhibition by serum was observed with the following strains Turkey 27, WS-57.7, WS-371A, X3 WS-309. Poor or no growth was observed with the strain Salvador.

### Example 6

### Agricultural Applications

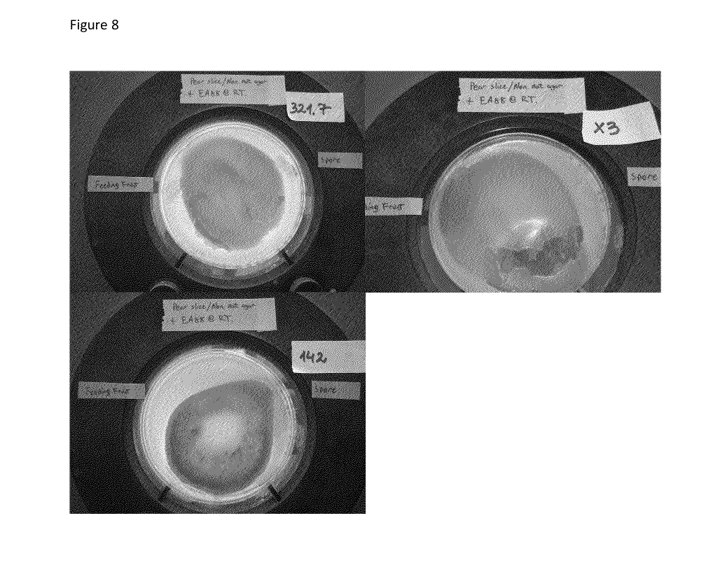

This example demonstrates the use of amoebae as antimicrobial agents in agricultural applications. In these methods, the amoebae are applied to plant surface to reduce or prevent microbial plant disease or spoilage. Results are shown in FIGS. 8 and 14-15.

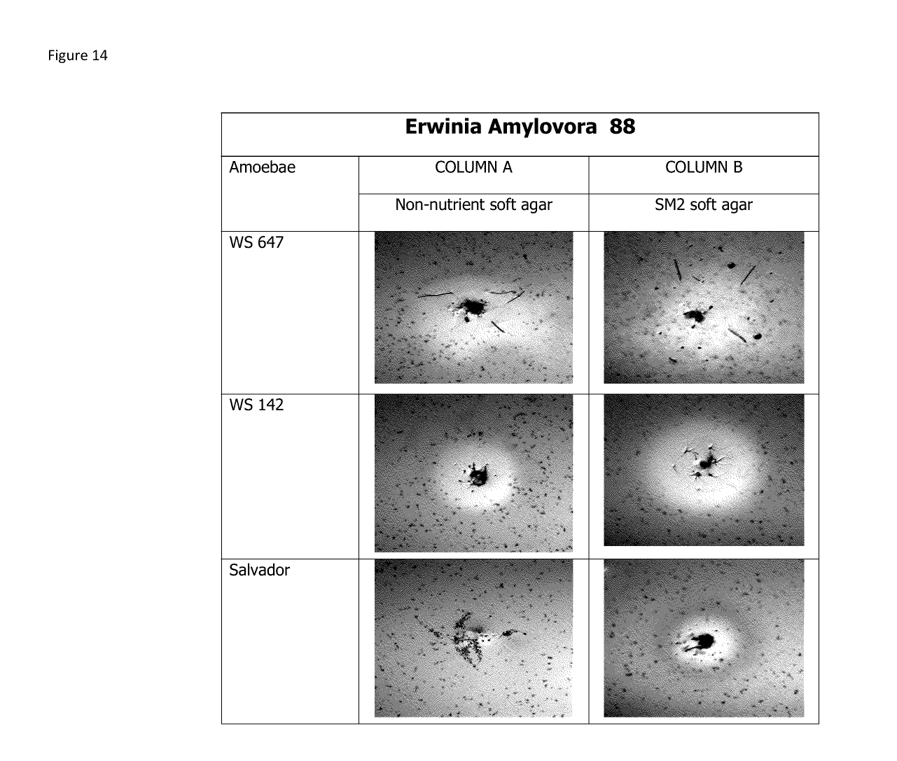

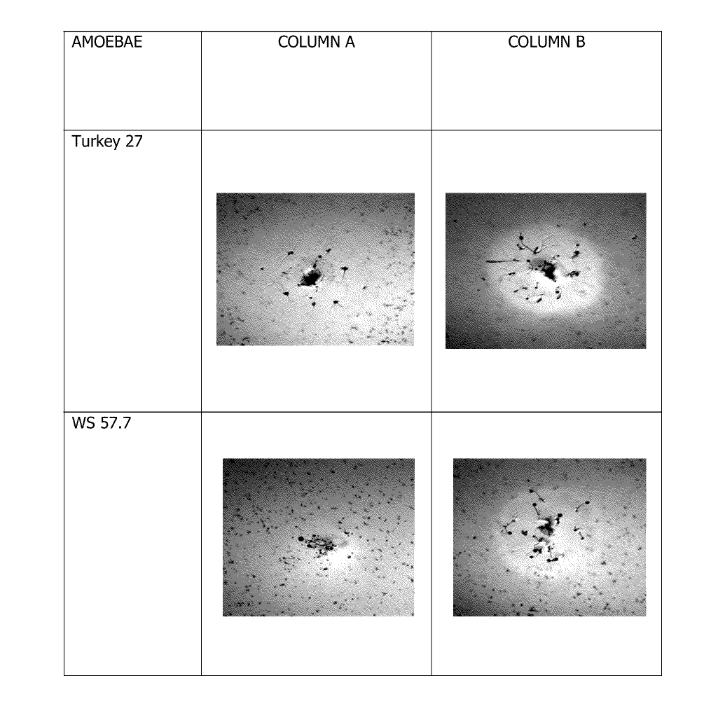

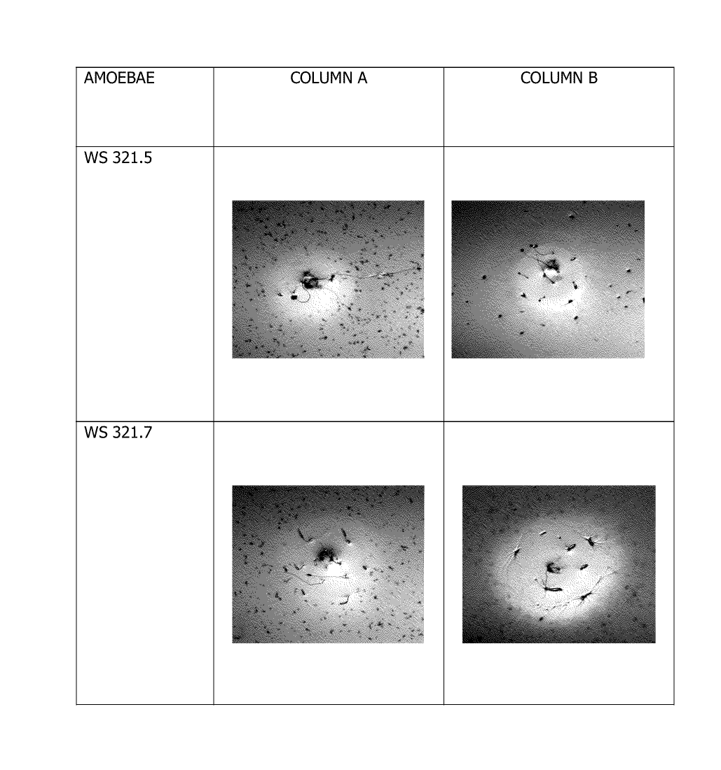

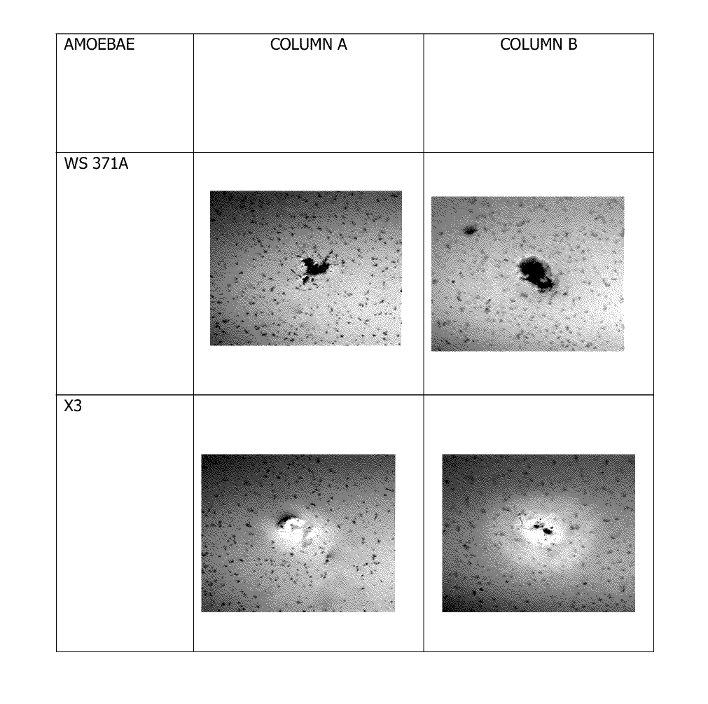

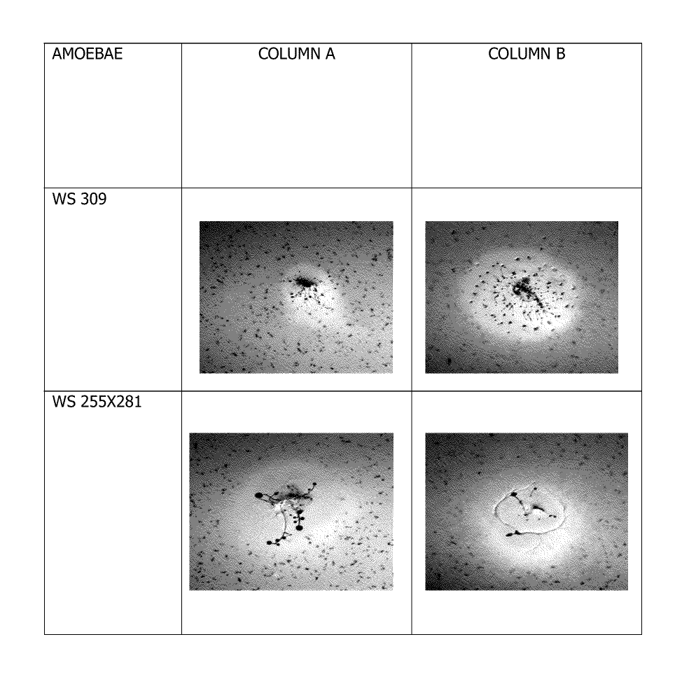

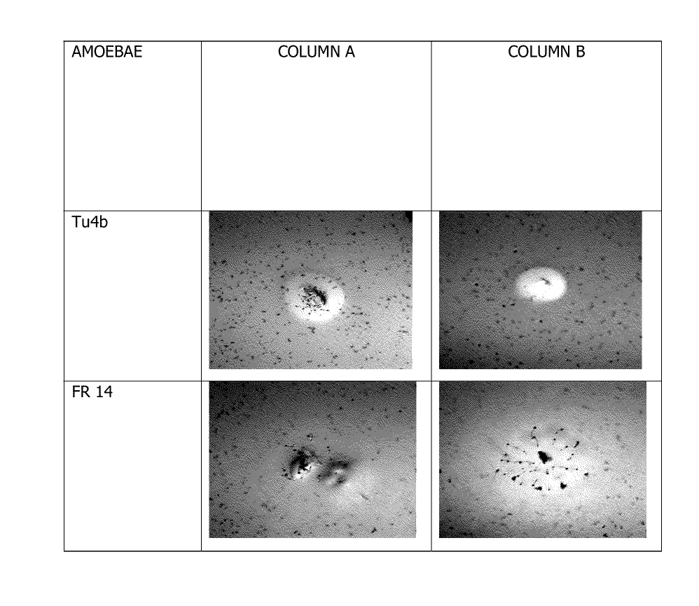

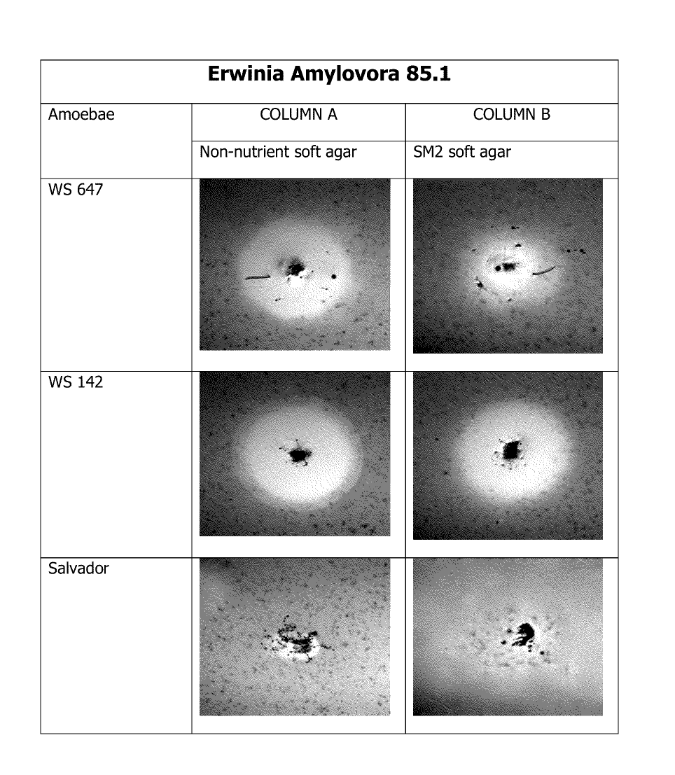

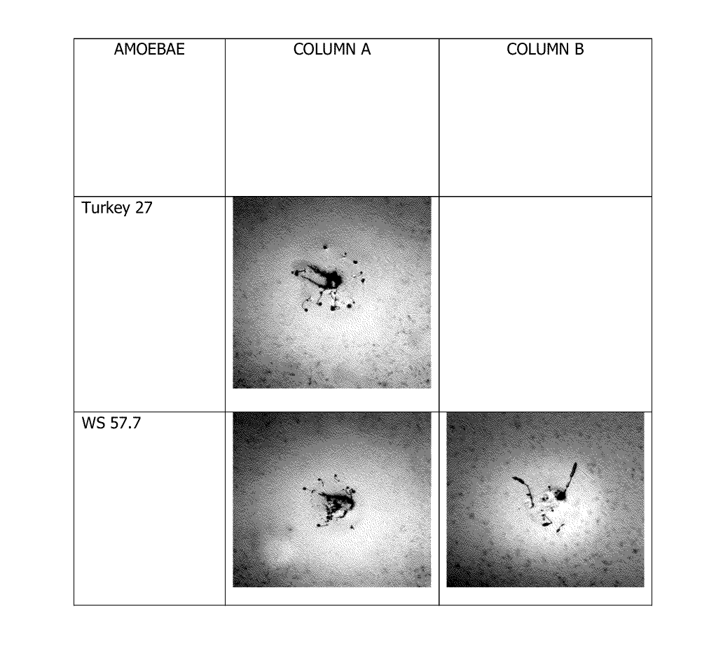

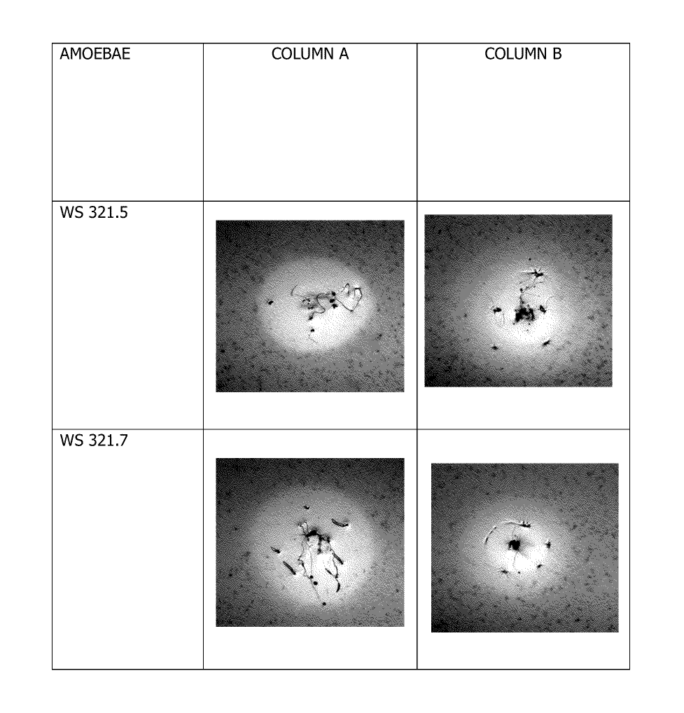

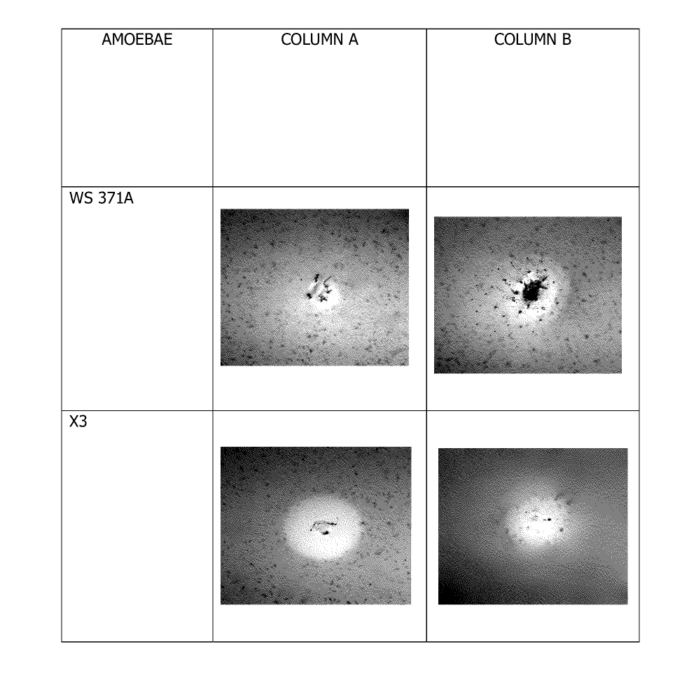

FIG. 14 shows feeding of amoebae on natural isolates of virulent strains of Erwinia amylovora (88, 85.1 and A97.1), a causative agent of Fire blight in pome fruits. Column A presents feeding and development of amoebae in the presence of Erwinia amylovora virulent isolates. Overnight culture of bacteria grown in SM2 medium was pelleted and resuspended in 0.9% sodium chloride for non-nutrient conditions and in SM2 liquid media for nutrient conditions (Column B). White irregular zone represents feeding front of free-living amoebae digesting bacteria inside this zone, aggregation, slugs and well-developed sporangia are visible as well. Pictures were taken using Olympus microscope/camera at 7-fold magnification.

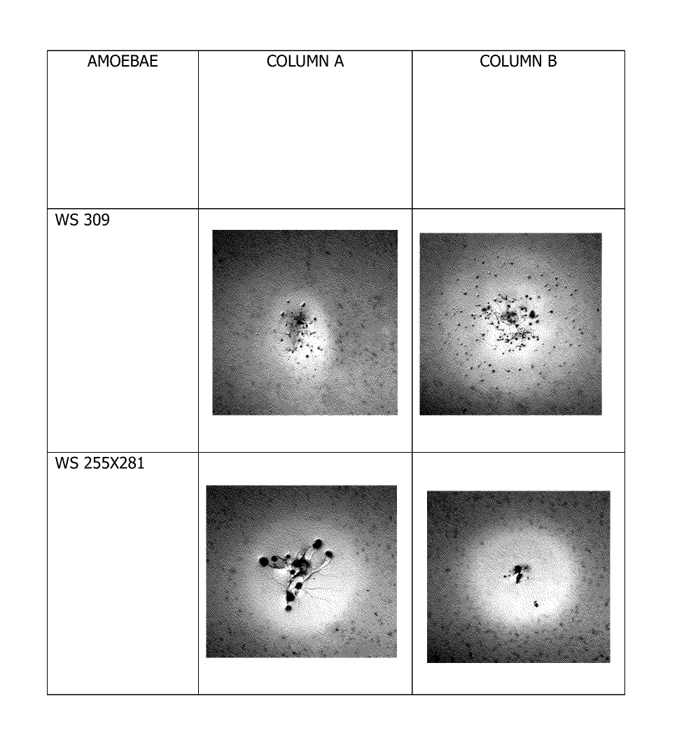

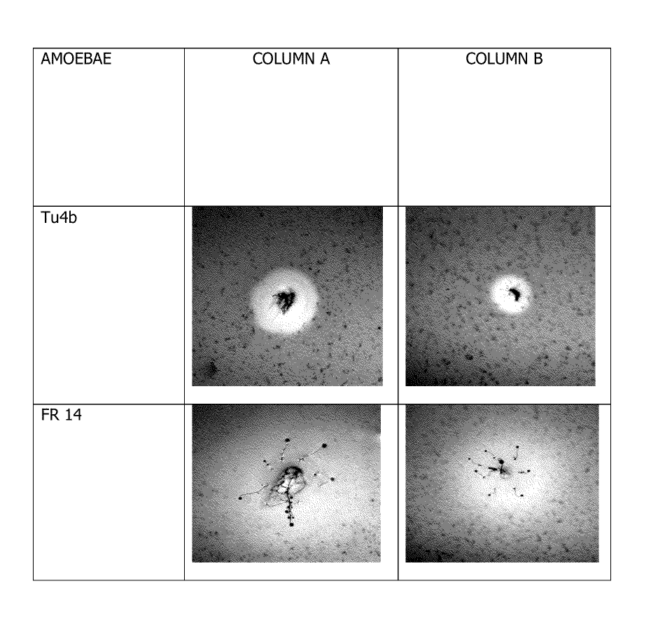

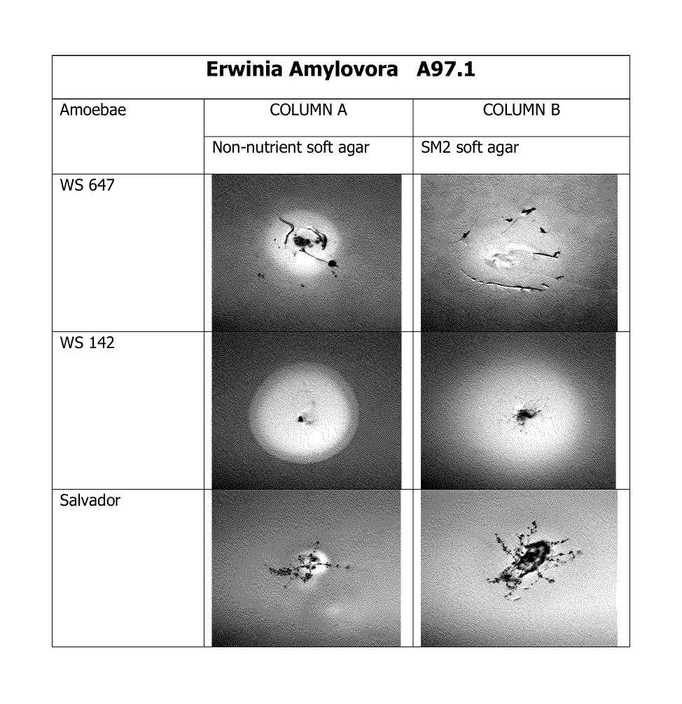

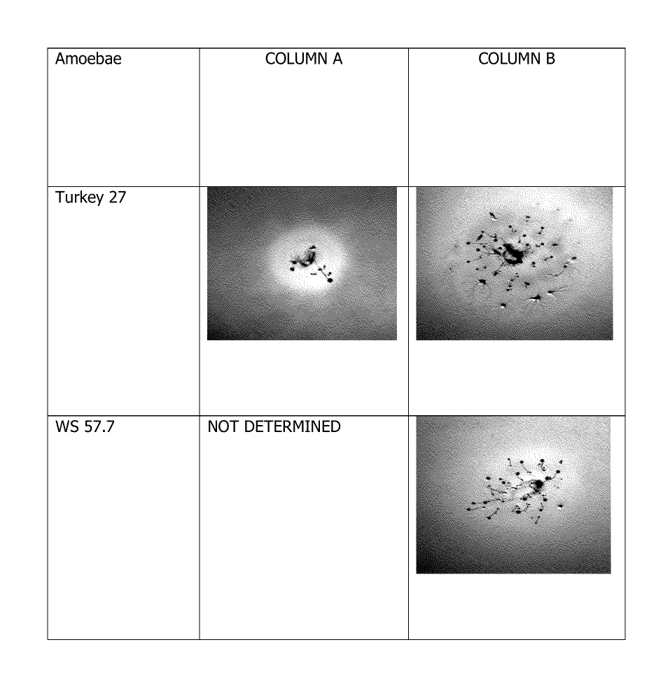

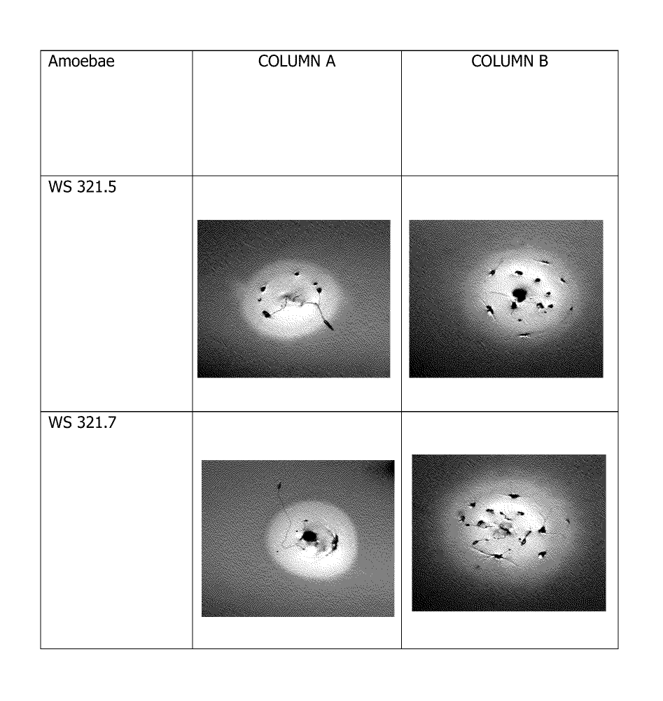

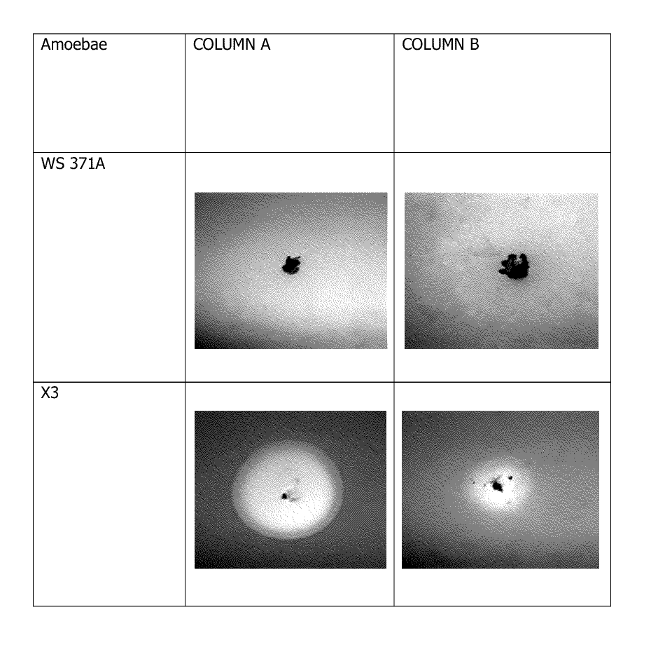

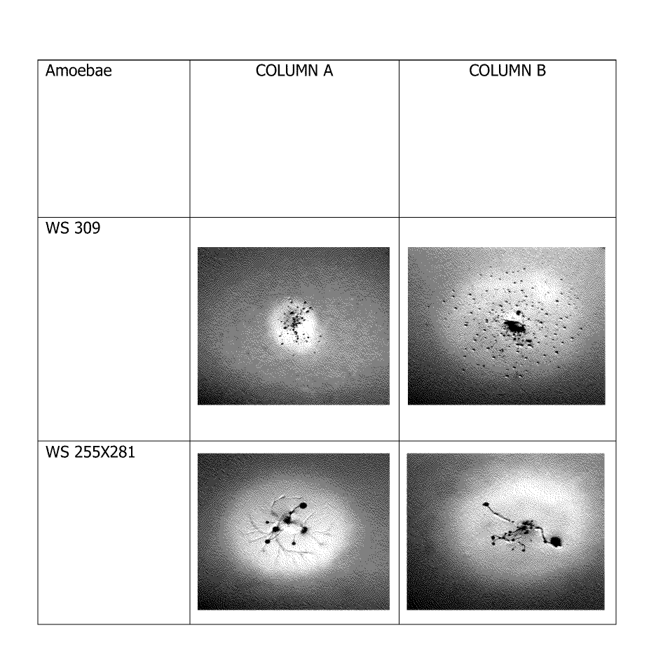

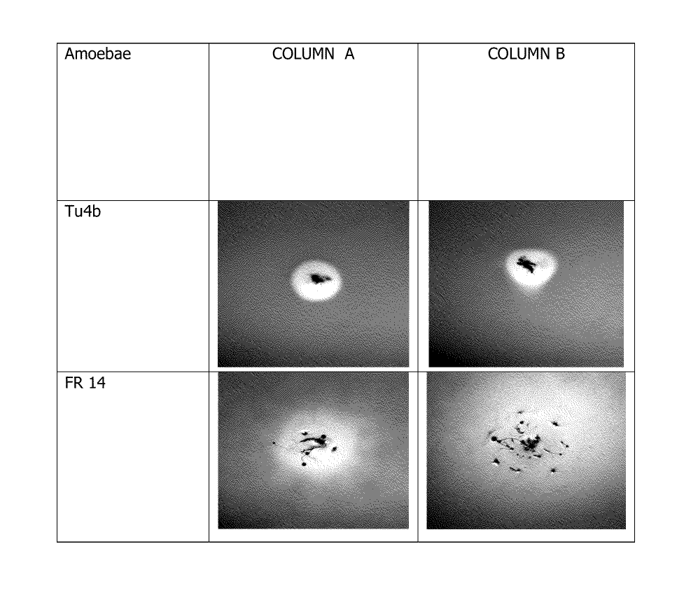

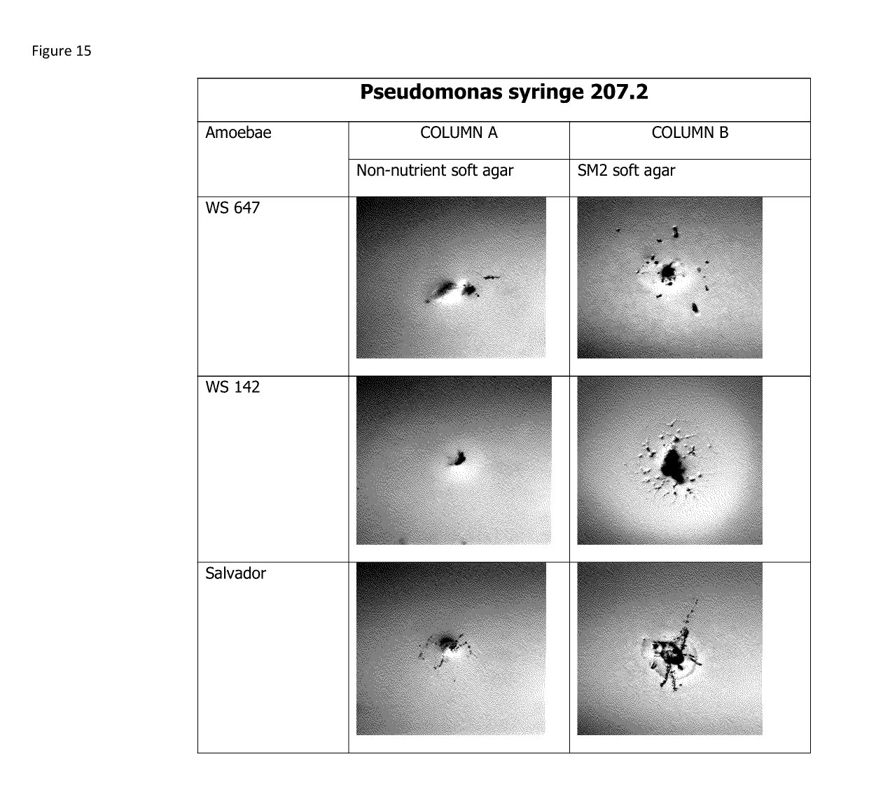

FIG. 15 shows feeding of amoebae on natural isolate virulent agent of bean disease Pseudomonas syringe 207.2. Column A shows zones of growth (or lack thereof) of amoebae on plates inoculated with P. syringiae 207.2. The plates were prepared by using the pathogen grown overnight in SM2 medium. Bacteria were peleted and resuspended in semi-solid agar containing MS2 nutrient agar (column A) or non-nutrient agar (Column B). Plates were incubated at room temperature and data was recorded after 48 hours (Clearing). Plates were photographed using a camera attached to an Olympus microscope at 8× magnification. Clearing zone shows feeding front of amoebae and structures inside are showing aggregation, slugs and mature fruiting bodies of amoebae with spores.

### Example 7

### Selected Dictyostelid Strains Feed on MRSA USA300 but not on Listeria monocytogenes

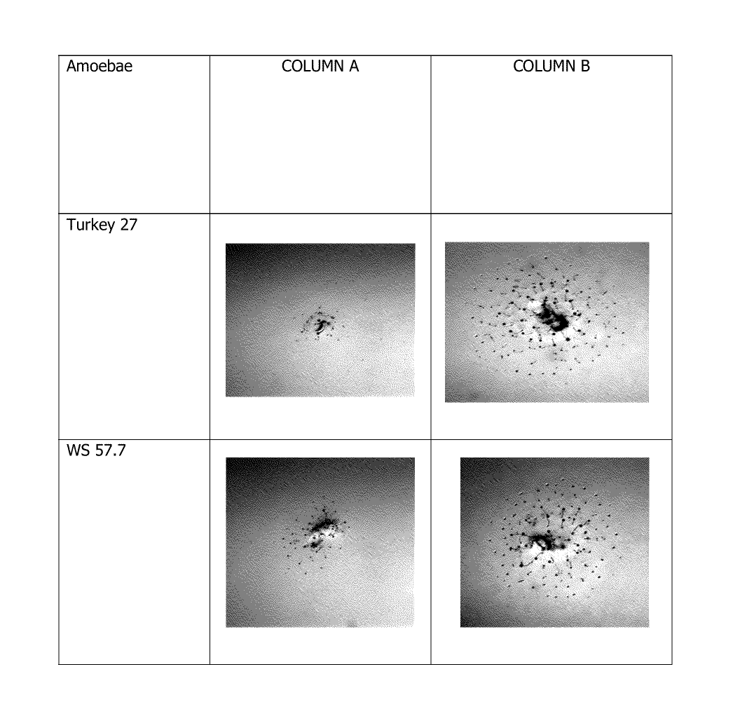

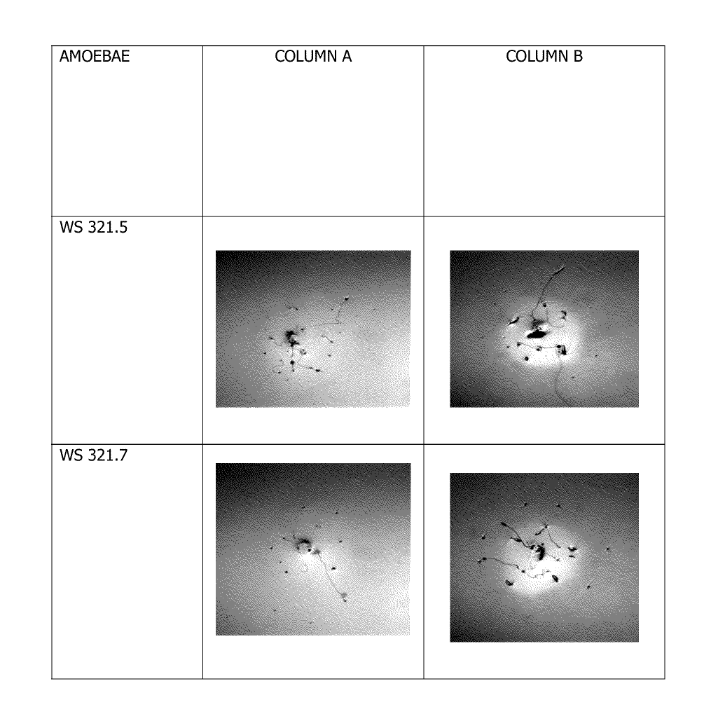

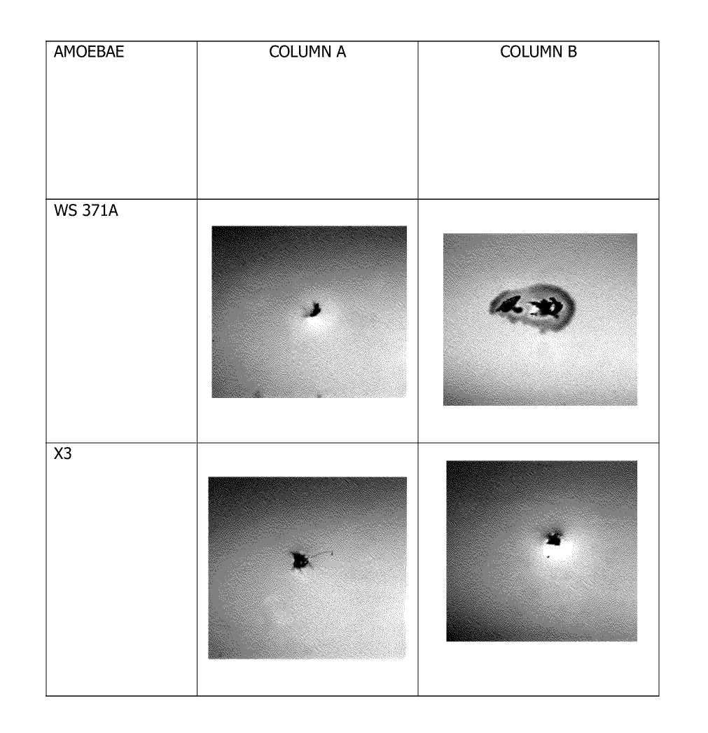

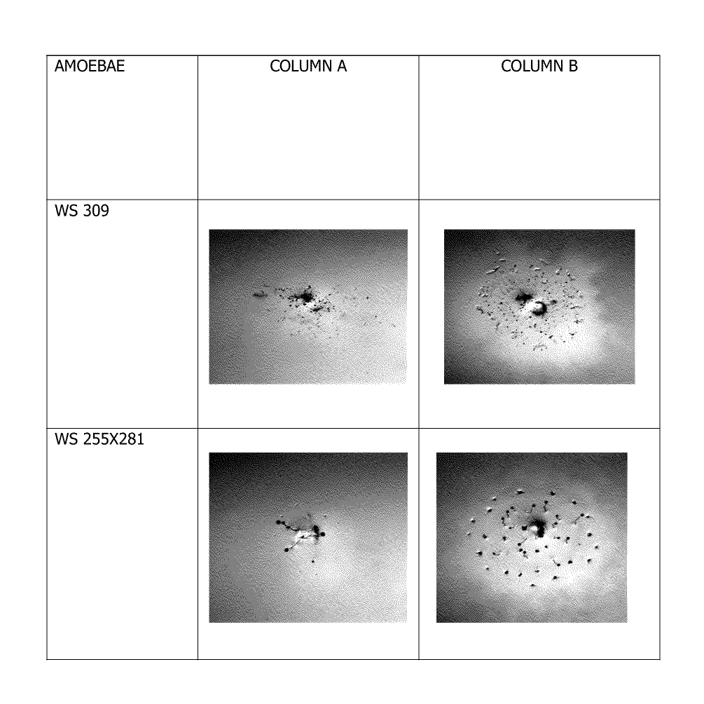

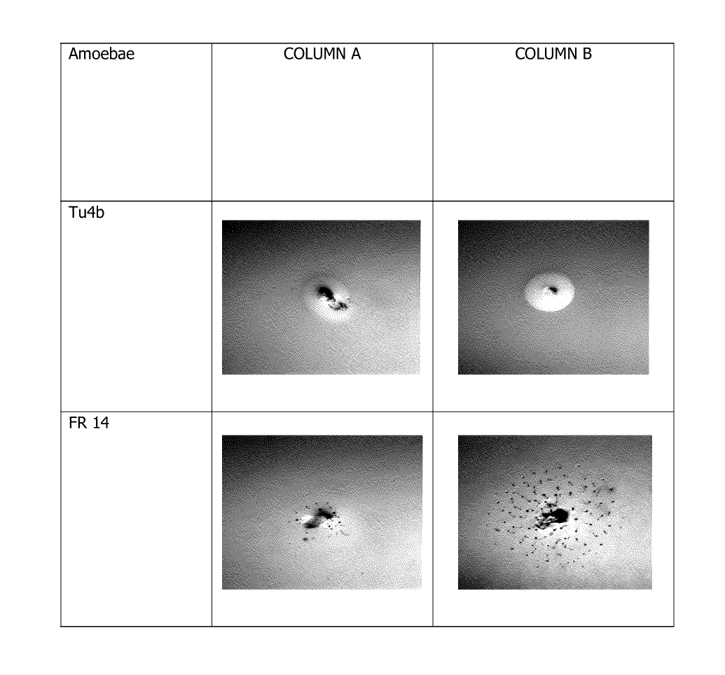

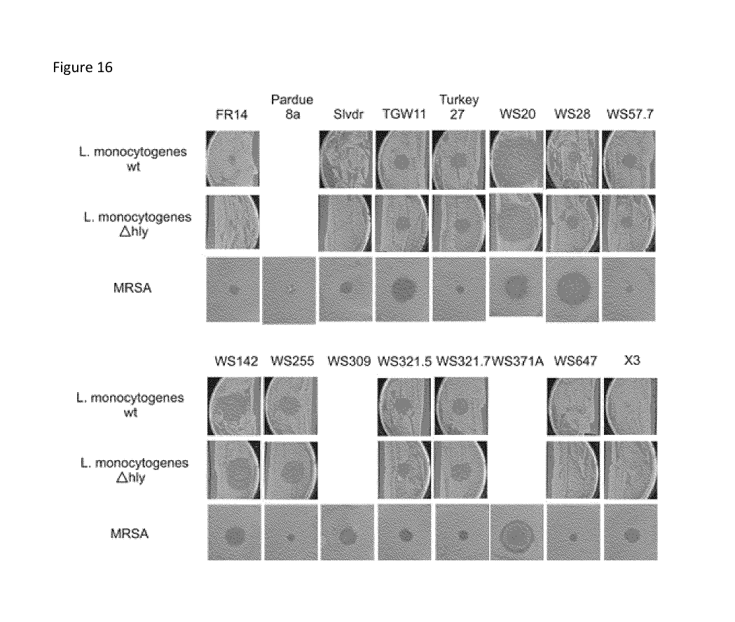

No published studies demonstrating that Dictyostelids feed on Listeria have been identified. Studies on Acanthamoeba (non Dictyostelid amoeba) have yielded mixed results with some data indicating they merely internalize the bacteria (Ly T M & Muller H E (1990) J Med Microbiol 33(1):51-54) while other data demonstrated Listeria were destroyed (Akya A, Pointon A, & Thomas C (2010) Microbiology 156(Pt 3):809-818). These discrepancies may have been influenced by factors such as the strains used (predator and/or prey), temperature, or other factors. Because two strains (AX3 and NC4) of a single species of myxamoebae, D. discoideum, have become well known as a model system for infection by some pathogenic bacteria, experiments were conducted to determine which myxamoebae can prey on Listeria. Side-by-side feeding of the same strains on lawns of MRSA USA300 were compared (FIG. 16). Lawns of one virulent and one avirulent Δhly (Jones S & Portnoy D A (1994) Infect Immun 62(12):5608-5613; Portnoy et al., (1988) J Exp Med 167(4):1459-1471) of L. monocytogenes were grown and seeded with thirteen Dictyostelid isolates. After 7 days of incubation at 25° C., three of the 13 isolates showed no indication of feeding on L. monocytogenes (both virulent and avirulent strains) (XA3, WS-647 and Salvador) and one strain fed poorly (FR14). The remaining nine strains (TGW11, Turkey 27, WS-20, WS-28, WS57.7, WS255, WS321.5, WS321.7 and WS371A) fed and produced sporangia on both the avirulent and the virulent strains. In contrast, AX3, WS647 and Salvador can feed on MRSA USA300.

### Example 8

### The Chemical Environment of the MRSA USA300 Colony-Biofilm Grown on SM/2 Agar is Conducive to the Spore Germination and Destruction by the Emerging and Multiplying Amoebae

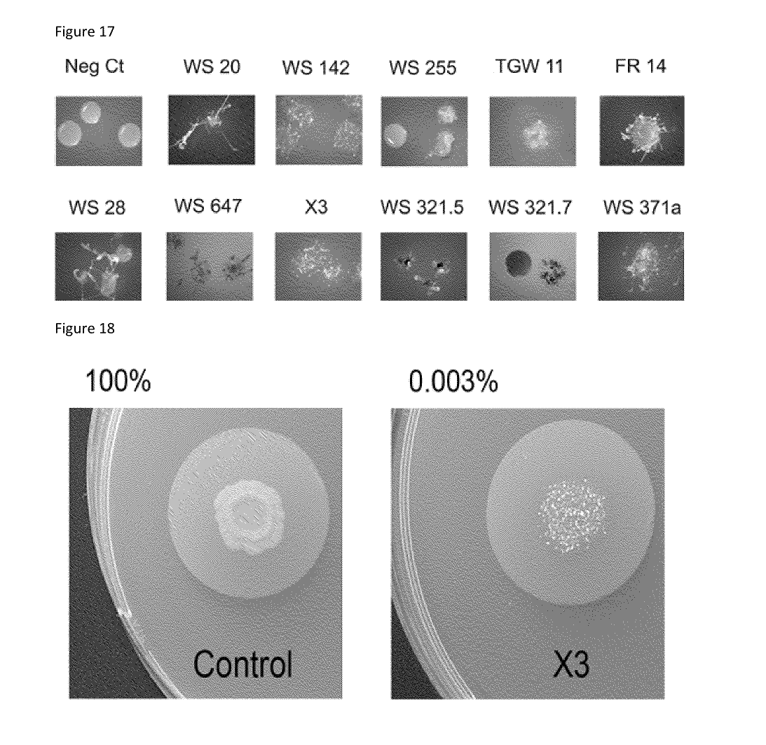

Spores were used to determine if the colony biofilm's chemical environment is conducive to spore germination. Dilutions of MRSA USA300 cultures were plated on SM/2 agar medium and incubated overnight at 37° C. The resulting biofilms colonies were overlaid with 1×10⁴ spores (in 10 l) per colony. Colonies and their destruction were observed daily and photographed. FIG. 17 shows end-stage macro-photographs of plates incubated for seven days. In the untreated control, a colony biofilm of MRSA USA300 can be seen. The fluffy structures that emerge as the biofilm destruction progresses are fruiting bodies of dictyostelid strains. Strain A X3 and WS-142 consumed colonies so cleanly that it was difficult to observe their remnants—quantitative data described in Example 9 support this conclusion.

### Example 9

### Chemical Environment of the MRSA USA300 Biofilm Grown on Polycarbonate Surface is Conducive to the Spore Germination and Destruction by the Emerging and Multiplying Amoebae

Spores were used to determine if the biofilm's chemical environment is conducive to spore germination; 2 polycarbonate membranes, 0.2 micron in pore size, resting on a SM2/2 agar plate were seeded with 10⁴ bacterial cells and grown overnight at 37° C. One biofilm patch was inoculated with 1×10⁴ spores of strain X3 and plates were incubated for 5 days at 25° C. Filters were observed daily and photographed. FIG. 18 shows end-stage macro-photographs of polycarbonate filters incubated for seven days. In the untreated control, a colony biofilm of MRSA USA300 can be seen. The fluffy structures that emerge as the biofilm destruction progresses are fruiting bodies of strain AX3. AX3 and WS-142 consumed biofilms formed on polycarbonate surface as cleanly as observed for the infected colony biofilms shown in Example 8; quantitative data support this conclusion. The filters were transferred with sterile forceps into a saline solution and vortexed until biofilms (observed/or not, macroscopically) were completely broken to individual cocci. Colony forming units were determined for each suspension. MRSA US300 cell death is expressed as the percentage of live cells detected in an uninfected control biofilm patch (5.5×10⁹ cfu). The numbers are averages of triplicate membranes.

Dictyostelids do not just kill bacterial cells encased in biofilms (e.g., by secreting an antibiotic substance). Rather, they physically destroy bacteria (ingest and digest) presented to them as biofilm-encased cells. This is evident when one considers the large amplification of dictyostelid cells needed to produce such a great number of multicellular fruiting bodies from the initial inoculum of 1×10⁴ spores.

### Example 10

### Adjusting Assay Parameters Allows for Biofilm Destruction by Free-Living Amoebae in Only a Few Hours

Methodology was modified to test whether manipulating the predator/prey ratios would demonstrate substantial destruction of bacterial biofilms in a matter of hours rather than days. For these studies strain AX3, which voraciously consumed bacteria in Example 9, was utilized. The choice of AX3 was based on its being the sole axenic strain of the seven presented here. Axenic dictyostelids can access nutrients by pinocytosis and be propagated to large numbers using synthetic medium rather than relying on bacteria for nourishment.

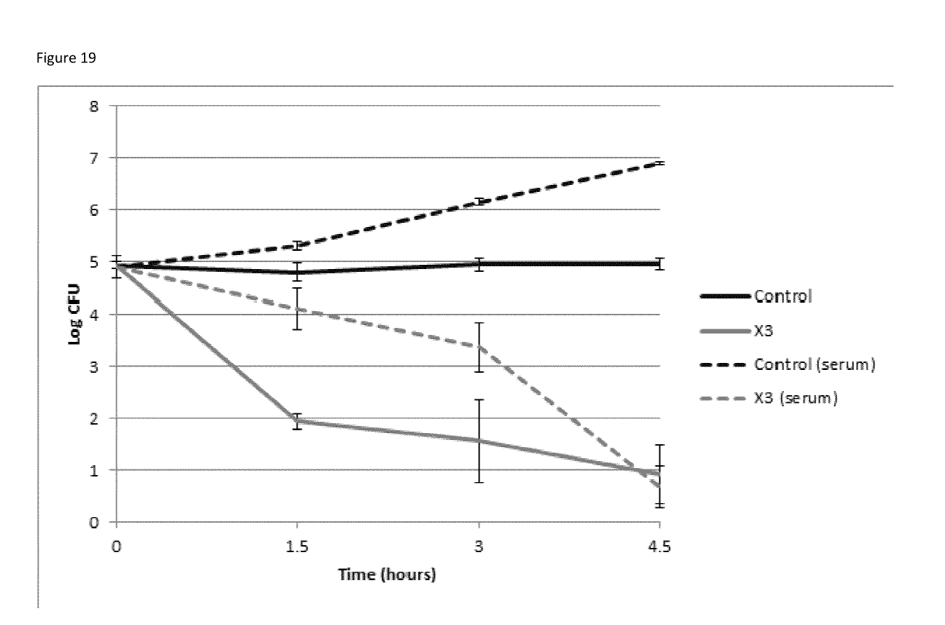

FIG. 19 directly compares feeding of strain AX3 on MRSA USA300 cells without and with serum addition. The graph shown in this figure demonstrates that AX3 amoebae can reduce MRSA-USA300 loads by several logᵢₙ in a few hours. For example, strain AX3 reduced the colony forming units (cfu) of the pathogen approximately by 4 log in 4.5 hours compared to the pre-treatment numbers; bacterial cfu were reduced (in presence of 25% porcine serum) approximately 6 log compared to the untreated controls. Virtually identical quantitative data were obtained for isolate WS-142 D. mucoroides. Of note is that MRSA USA300 does not grow at 25° unless the SM/2 medium is supplemented with serum.

### Example 11

### Polyspondillum palidum Salvador Feeds on Mrsa Usa300 Biofilm on Polycarbonate Surface at Human Body Temperature

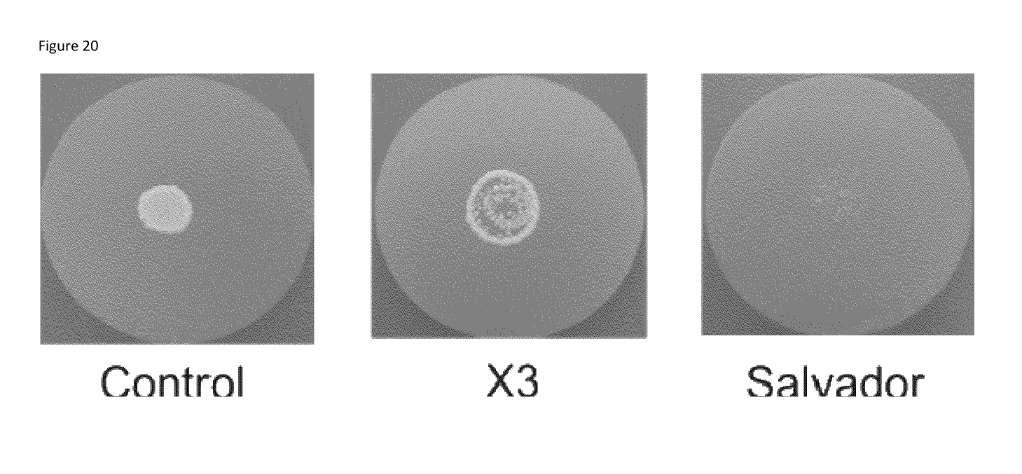

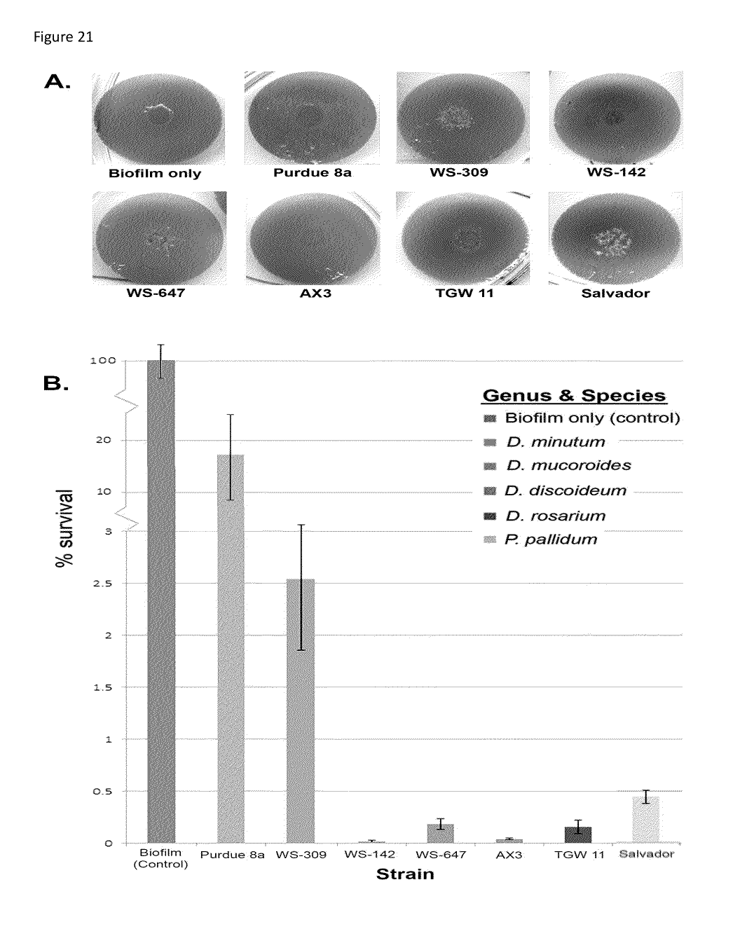

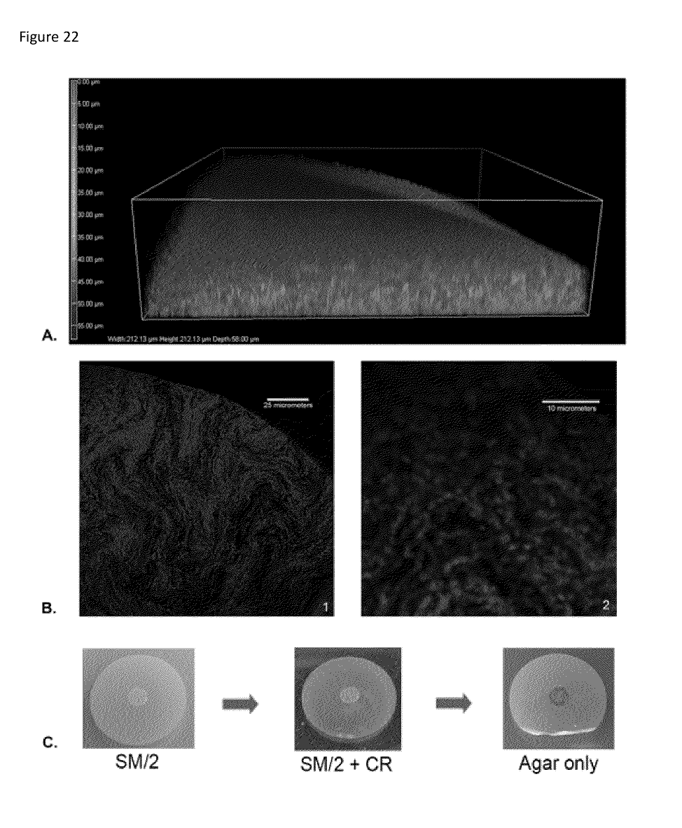

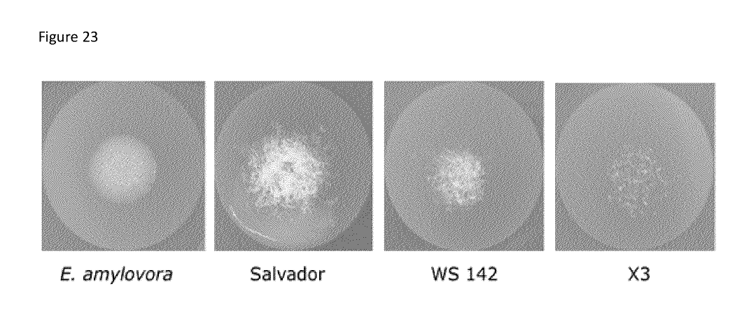

As pointed out in Example 3, not all Dictyostelid isolates are restricted to grow at the “typical” temperatures for wild type benchmark “soil” strains of 21° C.-25° C. (e.g., D. discoideumAX3 strain. Of 16 dictyostelid isolates observed feeding on the MRSA at 25°, (lawns pre-grown at 37° C. (FIG. 16), one tropical isolate (Polyspondillum pallidium, named Salvador) can also robustly feed at 37° C. on biofilm-encased cells of MRSA USA300 on polycarbonate surface (FIG. 20). 10 μl aliquots of planktonic bacteria (10⁵) were placed on the surface of polycarbonate filters resting on the SM/2 agar surface and before the drop was absorbed by the agar 10 μl aliquots of dictyostelid cells (10⁵) were applied. Photos were taken after incubation of plates at 37° C. for 18 hours. Whitish spots on the membrane with Salvador. represent sporangia produced by this strain upon the consumption of the MRSA USA300 biofilm.

### Example 12

### Polysphondylium violaceum WS-371a Feeds on Klebsiella pneumoniae Biofilm

Klebsiella pneumoniae is a pathogenic bacteria that is responsible for pneumoniae. Furthermore, this genus is of great importance as an agent of nosocomial infection (Ullman P R. (1998) Clinical Microbiology Reviews 11(4): 589-603). To test the idea that Polysphondylium violaceum WS-371a is able to feed on biofilms of Klebsiella pneumoniae, biofilms of Klebsiella pneumoniae were grown on glass coverslips, inoculated with Polysphondylium violaceum WS-371A amoebae, and incubated on a microscope stage at room temperature for roughly two days. It was observed that inoculation with this strain of amoeba drastically diminished the amount of biofilm on the cover slip. The time-lapse photography revealed that the amoebae devour the biofilm until it is essentially eliminated.

### Example 13

### Dictyostelium mucoroides Turkey 27 Feeds on Klebsiella pneumoniae Biofilm

Klebsiella pneumoniae is a pathogenic bacteria that is responsible for pneumoniae. Furthermore, this genus is of great importance as an agent of nosocomial infection (Ullman P R. (1998) Clinical Microbiology Reviews 11(4): 589-603). To test the idea that Dictyostelium mucoroides Turkey 27 is able to feed on biofilms of Klebsiella pneumoniae, biofilms of Klebsiella pneumoniae were grown on glass coverslips, inoculated with Dictyostelium mucoroides Turkey 27 amoebae, and incubated on a microscope stage at room temperature for roughly two days. It was observed that inoculation with this strain of amoeba drastically diminished the amount of biofilm on the cover slip. The time-lapse photography revealed that the amoebae devour the biofilm until it is essentially eliminated.

### Example 14

### Dictyostelium minutum Purdue 8a Poorly Feeds on Klebsiella pneumoniae Biofilm

Klebsiella pneumoniae is a pathogenic bacteria that is responsible for pneumoniae. Furthermore, this genus is of great importance as an agent of nosocomial infection (Ullman P R. (1998) Clinical Microbiology Reviews 11(4): 589-603). To test the idea that Dictyostelium minutum Purdue 8a is able to feed on biofilms of Klebsiella pneumoniae, biofilms of Klebsiella pneumoniae were grown on glass coverslips, inoculated with Purdue 8a amoebae, and incubated on a microscope stage at room temperature for roughly two days. It was observed that inoculation with this strain of amoeba drastically diminished the amount of biofilm on the cover slip. Time-lapse photography revealed that the amoebae devour the biofilm until it is essentially eliminated.

### Example 15

### Polyspondillum palidum Salvador Feeds on Klebsiella pneumoniae Biofilm

Klebsiella pneumoniae is a pathogenic bacteria that is responsible for pneumoniae. Furthermore, this genus is of great importance as an agent of nosocomial infection (Ullman P R. (1998) Clinical Microbiology Reviews 11(4): 589-603). To test the idea Polyspondillum palidum Salvador is able to feed on biofilms of Klebsiella pneumoniae, biofilms of Klebsiella pneumoniae were grown on glass coverslips, inoculated with Polyspondillum palidum Salvador amoebae, and incubated on a microscope stage at room temperature for roughly two days. It was observed that inoculation with this strain of amoeba drastically diminished the amount of biofilm on the cover slip. Time-lapse photography revealed that the amoebae devour the biofilm until it is nearly eliminated.

### Example 16

### Dictyostelium rosarium TGW-11 Feeds on Klebsiella pneumoniae Biofilm

Klebsiella pneumoniae is a pathogenic bacteria that is responsible for pneumoniae. Furthermore, this genus is of great importance as an agent of nosocomial infection (Ullman P R. (1998) Clinical Microbiology Reviews 11(4): 589-603). To test the idea that Dictyostelium rosarium TGW-11 is able to feed on biofilms of Klebsiella pneumoniae, biofilms of Klebsiella pneumoniae were grown on glass coverslips, inoculated with Dictyostelium rosarium TGW-11 amoebae, and incubated on a microscope stage at room temperature for roughly two days. It was observed that inoculation with this strain of amoeba drastically diminished the amount of biofilm on the cover slip. Time-lapse photography revealed that the amoebae devour the biofilm until it is essentially eliminated.

### Example 17

### Dictyostelium mucoroides WS-142 Feeds on Klebsiella pneumoniae Biofilm

Klebsiella pneumoniae is a pathogenic bacteria that is responsible for pneumoniae. Furthermore, this genus is of great importance as an agent of nosocomial infection (Ullman P R. (1998) Clinical Microbiology Reviews 11(4): 589-603). To test the idea that Dictyostelium mucoroides WS-142 is able to feed on biofilms of Klebsiella pneumoniae, biofilms of Klebsiella pneumoniaee were grown on glass coverslips, inoculated with Dictyostelium mucoroides WS-142 amoebae, and incubated on a microscope stage at room temperature for roughly two days. It was observed that inoculation with this strain of amoeba drastically diminished the amount of biofilm on the cover slip. Time lapse photography reveals that the amoebae devour the biofilm until it is essentially eliminated.

### Example 18

### Dictyostelium discoideum WS-647 Feeds on Klebsiella pneumoniae Biofilm

Klebsiella pneumoniae is a pathogenic bacteria that is responsible for pneumoniae. Furthermore, this genus is of great importance as an agent of nosocomial infection (Ullman P R. (1998) Clinical Microbiology Reviews 11(4): 589-603). To test the idea that Dictyostelium discoideum WS-647 is able to feed on biofilms of Klebsiella pneumoniae, biofilms of Klebsiella pneumoniae were grown on glass coverslips, inoculated with Dictyostelium discoideum WS-647 amoebae, and incubated on a microscope stage at room temperature for roughly two days. It was observed that inoculation with this strain of amoeba drastically diminished the amount of biofilm on the cover slip. Time lapse photography revealed that the amoebae devour the biofilm until it is nearly eliminated.

### Example 19

### Dictyostelium sphaerocephalum FR-14 Feeds on Klebsiella pneumoniae Biofilm

Klebsiella pneumoniae is a pathogenic bacteria that is responsible for pneumoniae. Furthermore, this genus is of great importance as an agent of nosocomial infection (Ullman P R. (1998) Clinical Microbiology Reviews 11(4): 589-603). To test the idea that FR-14 is able to feed on biofilms of Klebsiella pneumoniae, biofilms of Klebsiella pneumoniae were grown on glass coverslips, inoculated with FR-14 amoebae, and incubated on a microscope stage at room temperature for roughly two days. It was observed that inoculation with this strain of amoeba drastically diminished the amount of biofilm on the cover slip. Time lapse photography revealed that the amoebae devour the biofilm until it is greatly diminished.

### Example 20

### Dictyostelium discoideum WS-647 Feeds on Pseudomonas aeruginosa

Pseudomonas aeruginosa is a ubiquitous bacteria known to colonize the urinary tract, lungs, and kidneys and often lead to sepsis and death. Furthermore, this bacterium is known to thrive on surfaces such as catheters, potentially as a biofilm (Balcht, Aldona & Smith, Raymond (1994). Pseudomonas aeruginosa: Infections and Treatment. Informa Health Care. pp. 83-84). Additionally, the biofilm formed by this bacterium is thought to resist protozoan grazing (Kjelleberg S., Environmental Microbiology (2006) 7(10): 1593-1601). To test the idea that Dictyostelium discoideum WS-647 amoebae are able to feed on Pseudomonas aergutinosa biofilm, biofilms of Pseudomonas aeruginosa were grown on SM/2 agar, inoculated with Dictyostelium discoideum WS-647 amoebae, and incubated at room temperature for roughly two days. It was observed that inoculation with this strain of amoeba is responsible for diminished biofilm on the agar surface.

### Example 21

### Dictyostelium mucoroides WS-20 Feeds on Pseudomonas aeruginosa

Pseudomonas aeruginosa is a ubiquitous bacteria known to colonize the urinary tract, lungs, and kidneys and often lead to sepsis and death. Furthermore, this bacterium is known to thrive on surfaces such as catheters, potentially as a biofilm (Balcht, Aldona & Smith, Raymond (1994). Pseudomonas aeruginosa: Infections and Treatment. Informa Health Care. pp. 83-84). Additionally, the biofilm formed by this bacterium is thought to resist protozoan grazing (Kjelleberg S., Environmental Microbiology (2006) 7(10): 1593-1601). To test the idea that Dictyostelium mucoroides WS-20 amoebae are able to feed on Pseudomonas aergutinosa biofilm, biofilms of Pseudomonas aeruginosa were grown on SM/2 agar, inoculated with Dictyostelium mucoroides WS-20 amoebae, and incubated at room temperature for roughly 5 days. It was observed that inoculation with this strain of amoeba is responsible for diminished biofilm on the agar surface.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

We claim:

1. A method of treating a biofilm accumulation, comprising:
contacting said biofilm with a composition comprising one or more species of purified amoebae.

2. The method of claim 1, wherein said biofilm is produced by a microbial organism selected from the group consisting of bacteria, protozoa, amoeba, and fungi.

3. The method of claim 2, wherein said microbial organisms are pathogenic.

4. The method of claim 1 wherein said biofilm is in or on a surface.

5. The method of claim 1, wherein said biofilm is in or on a subject.

6. The method of claim 6, wherein said microorganism is in a wound.

7. The method of claim 7, wherein said wound is at a temperature above the normal body temperature of said subject.

8. The method of claim 7, wherein said wound is hypoxic.

9. The method of claim 6, wherein said microorganism is on a mucus membrane of said subject.

10. The method of claim 5, wherein said microorganism is in an organ or tissue of said subject.

11. The method of claim 1, wherein said microorganism is in or on a plant.

12. The method of claim 1, wherein said composition comprises two or more species of amoebae.

13. The method of claim 1, wherein said amoebae are selected from the group consisting of Dictyostelium discoideum (WS-28 and WS-647 and X3); D. minutum (Purdue 8a); D. mucoroides (Turkey 27, WS-20, WS-142, WS-255); D. mucoroides complex (WS-309); D. purpureum (WS-321.5 and WS-321.7); D. rosarium (TGW-11); D. sphaerocephalum (FR-14); Polysphondylium pallidum (Salvador); P. violaceum (WS-371a) and unknown isolate (Tu-4-b).

14. The method of claim 1, wherein said composition further comprises a non-amoebae anti-microbial agent.

15. The method of claim 1, wherein said composition is a pharmaceutical agent.

16. The method of claim 4, wherein said surface is a shower drain, water pipe, sewage pipe, food preparation surface, gas or oil pipeline, medical device, contact lens, or ship hull.

17. The method of claim of claim 1 wherein the biofilm is located on the surface at a facility selected from the group consisting of hospitals, laboratories, water treatment facilities, sewage treatment facilities, dental and/or medical offices, water distribution facilities, nuclear power plant, pulp or paper mill, air and/or water handling facility, pharmaceutical manufacturing facility, and dairy manufacturing facility.

18. A method of treating a subject infected with a biofilm, comprising: contacting a subject infected with a biofilm with a pharmaceutical composition comprising one or more species of amoebae.

19. The method of claim 17, wherein said subject is a human.

20. A pharmaceutical composition, comprising:
a) one or more species of amoebae; and
b) a pharmaceutically acceptable carrier.
